SEQUENCE LISTING (6 sequences)

SEQ ID NO: 1 - DNA, 1528 bp, Barnesiella intestinihominis
  source 1..1528: mol_type = other DNA
cgaagagtttgatcctggctcaggatgaacgctagcgacaggcctaacacatgcaagtcgaggggcagcggagaggtagcaatacctttgccggcgaccggcgcacgggtgagtaacacgtatgcaatccacctgtaacagggggataacccggagaaatccggactaataccccataatatgggctctccgcatggagggtccattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaattttcgggctcaacccggactgtgccgttgaaactggcgagctagagtgcacaagaggcaggcggaatgcgtggtgtagcggtgaaatgcatagatatcacgcagaaccccgattgcgaaggcagcctgctagggtgcgacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatatatatgaaagtatatagctagcaatagtcacctgcgaggtgctgcatggttgtcgtcagctcgtgccgtgaggtgtcggcttaagtgccataacgagcgcaacccctatcgacagttactaacgggtcaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaaggtgcggctggaacacctccttt

SEQ ID NO: 2 - DNA, 1437 bp, Barnesiella intestinihominis
  source 1..1437: mol_type = other DNA
cggcgaccggcgcacgggtgagtaacacgtatgcaatccacctgtaacagggggataacccggagaaatccggactaataccccataatatgggctctccgcatggagggtccattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgcgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggaatgtgccgttgaaactggcgagctagagtgcacaagaggcaggcggaatgcgtggtgtagcggtgaaatgcatagatatcacgcagaaccccgattgcgaaggcagcctgctagggtgcgacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatatatatgaaagtatatagctagcaatagtcacctgcgaggtgctgcatggttgtcgtcagctcgtgccgtgaggtgtcggcttaagtgccataacgagcgcaacccctatcgacagttactaacgggtcaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaaggtgcggctggaacacctccttt

SEQ ID NO: 3 - DNA, 1521 bp, Alistipes onderonkii
  source 1..1521: mol_type = other DNA
atggagagtttgatcctggctcaggatgaacgctagcggcaggcctaacacatgcaagtcgaggggcatcacgaggtagcaatactttggtggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgagaaaccttacccgggcttgaaagttactgacgattctggaaacaggatttccctttggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctactccgtgaggagatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtaactggggctaagtcgtaacaaggtagccgtaccggaaggtgcggctggaacacctccttt

SEQ ID NO: 4 - DNA, 1526 bp, Bacteroides finegoldii
  source 1..1526: mol_type = other DNA
atgaagagtttgatcctggctcaggatgaacgctagctacaggcttaacacatgcaagtcgaggggcagcattttagtttgcttgcaaactaaagatggcgaccggcgcacgggtgagtaacacgtatccaacctgccgataactcagggatagcctttcgaaagaaagattaatacctgatggcataggattatcgcatgataatcctattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatggacgggagtctgaaccagccaagtagcgtgaaggatgactgccctatgggttgtaaacttcttttatacgggaataaagtgatccacgtgtgggtttttgtatgtaccgtatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgatactggcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacggtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattacatttgaataatctggaaacaggttagccgtaaggcaaatgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcgaggagcgtcctagggtaaaactggtaattggggctaagtcgtaacaaggtagccgtaccggaaggtgcggctggaacacctccttt

SEQ ID NO: 5 - DNA, 1525 bp, Bacteroides vulgatus
  source 1..1525: mol_type = other DNA
atgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagttcacatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttataaaggaataaagtcgggtatgtatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacagcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctccttt

SEQ ID NO: 6 - DNA, 1528 bp, Oscillibacter sp.
  source 1..1528: mol_type = other DNA
tatagagagtttgatcctggctcaggacgaacgctggcggcgtgcttaacacatgcaagtcgaacggagcacccttgattgaggtttcggccaaatgataggaatgcttagtggcggactggtgagtaacgcgtgaggaacctgcctttcagagggggacaacagttggaaacgactgctaataccgcatgacgcattttgatcgcatggtcgaaatgccaaagatttatcgctgaaagatggcctcgcgtctgattagatagttggcggggtaacggcccaccaagtcgacgatcagtagccggactgagaggttgaccggccacattgggactgagatacggcccagactcctacgggaggcagcagtggggaatattgggcaatgggcgcaagcctgacccagcaacgccgcgtgaaggaagaaggctttcgggttgtaaacttcttttaagtgggaagagtagaagacggtaccacttgaataagccacggctaactacgtgccagcagccgcggtaatacgtaggtggcaagcgttgtccggatttactgggtgtaaagggcgtgcagccgggcatgcaagtcagatgtgaaatctcagggcttaaccctgaaactgcatttgaaactgtatgtcttgagtgccggagaggtaatcggaattccttgtgtagcggtgaaatgcgtagatataaggaagaacaccagtggcgaaggcggattactggacggtaactgacggtgaggcgcgaaagcgtggggagcgaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgaagcaacgcgaagaaccttaccaggtcttgacatcctgctaacgaagtagagatacattaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgacctgggctacacacgtaatacaatggcggtcaacaaagggatgcaaagccgcgaggcagagcgaaccccaaaaagccgtcccagttcggatcgcaggctgcaacccgcctgcgtgaagtcggaatcgctagtaatcgtgggtcagcataccacggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgttcgagaacgagcggctggatcacctccttt

(19) United States
(12) Patent Application Publication
(10) Pub. No.: US 2025/0017981 A1
(43) Pub. Date: Jan. 16, 2025

(54) COMPOSITIONS FOR METABOLIC HEALTH

(71) Applicant: INTERNATIONAL N&H DENMARK APS, KONGENS LYNGBY (DK)

(72) Inventors: Ritesh Kumar, Wilmington, DE (US); Helene Kane, Woodbury, NJ (US); Qiong Wang, Wilmington, DE (US); Henrik Max Jensen, Brabrand (DK); Pia Tuulikki Rasinkangas, Helsinki (FI); Arthur Ouwehand, Inga (FI); Ashley Hibberd, St. Louis, MO (US); Buffy Stahl, Oregon, WI (US); Sofia Forssten, Tenala (FI); Oliver Hasselwander, Surrey (GB); Hye-Sook Kim, Hockessin, DE (US); Pierre Rouviere, Wilmington, DE (US)

(21) Appl. No.: 18/712,074

(22) PCT Filed: Nov. 22, 2022
(86) PCT No.: PCT/US22/80311

§ 371 Date: May 21, 2024

Related U.S. Application Data
(60) Provisional application No. 63/282,105, filed on Nov. 22, 2021.

(51) Int. Cl. A61K 35/741

(52) U.S. Cl. CPC A61K 35/741

(57) ABSTRACT

Provided herein, inter alia, are compositions comprising one or more biologically pure strains of bacteria as well as methods of making and using the same to treat and/or prevent one or more obesity related disorders in a subject in need thereof.

### CROSS REFERENCE TO RELATED APPLICATION

This application claims priority to U.S. Provisional Application No. 63/282,105, filed on Nov. 22, 2021, the disclosure of which is incorporated by reference herein in its entirety.

### INCORPORATION BY REFERENCE

The sequence listing provided in the file named 20221121_NB42000-WO-PCT_SequenceListing.xml with a size of 15 KB which was created on Nov. 21, 2022 and which is filed herewith, is incorporated by reference herein in its entirety.

### FIELD OF THE INVENTION

Provided herein, inter alia, are bacterial compositions useful for improving metabolic health in subjects as well as methods for making and using the same.

### BACKGROUND

The human gastrointestinal tract contains a complex and diverse ecosystem of microorganisms. Intestinal bacteria are not only commensal, but they also undergo a symbiotic co-evolution with their host. The interaction between gut microbiota and host is complex. Beneficial intestinal bacteria have numerous important functions and they directly or indirectly affect various physiological functions in the host, e.g., they provide nutrients to their host, prevent infections caused by intestinal pathogens, and modulate a normal immunological response. It is established that imbalance in the microbiota composition results in various disease states in the host. Therefore, modification of the intestinal microbiota in order to achieve, restore, and maintain favorable balance in the ecosystem, as well as the activity of microorganisms present in the gastrointestinal tract, is necessary for maintaining and improving the health condition of the host.

First-generation probiotics are live microorganisms mainly derived from the genera Lactobacillus and Bifidobacterium, which are often minor constituents of the digestive tract or originate from use as dairy starter cultures. Traditionally, first-generation probiotics are mainly targeted at gut and immune health. Some, such as B. lactis B420, have been shown exert beneficial activity also in relation to metabolic health, e.g., in reduction in body fat mass and some improvement for blood glucose and insulin. However, current first-generation probiotics do not seem to provide an optimal solution with respect to glucose and insulin metabolism, i.e., as potential treatments or preventative agents for type 2 diabetes, pre-diabetes, or metabolic syndrome.

What is needed, therefore, are additional microorganisms identified based on their natural occurrence in the digestive tract of metabolically healthy individuals and which have been selected based on their ability to maintain and optimize metabolic health and prevent disease.

The subject matter disclosed herein addresses these needs and provides additional benefits as well.

### SUMMARY

Provided herein, inter alia, are compositions comprising one or more biologically pure strains of bacteria and methods of making and using the same to treat and/or prevent one or more obesity related disorders, such as, but not limited to, obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease in a subject in need thereof.

Accordingly, in some aspects, provided herein is a composition comprising at least one or more of (a) a biologically pure strain of Barnesiella intestinihominis; (b) a biologically pure strain of Alistipes onderdonkii; (c) a biologically pure strain of Bacteroides finegoldii; (d) a biologically pure strain of Bacteroides vulgatus; and/or (e) a bacterial strain having a 16S ribosomal RNA sequence displaying at least 97.0% sequence similarity to a 16S ribosomal RNA sequence of an Oscillibacter sp. deposited at the German Collection of Microorganisms and Cell Cultures (DSM) under number DSM 34011. In some embodiments, the composition comprises (a)(i) a bacterial strain having a 16S ribosomal RNA sequence displaying at least 97.0% sequence similarity to a 16S ribosomal RNA sequence of Barnesiella intestinihominis deposited at DSM under number DSM 34032; and/or (ii) a bacterial strain having a 16S ribosomal RNA sequence displaying at least 97.0% sequence similarity to a 16S ribosomal RNA sequence of Barnesiella intestinihominis deposited at DSM under number DSM 34012; (b) a bacterial strain having a 16S ribosomal RNA sequence displaying at least 97.0% sequence similarity to a 16S ribosomal RNA sequence of Alistipes onderdonkii deposited at DSM under number DSM 34033; (c) a bacterial strain having a 16S ribosomal RNA sequence displaying at least 97.0% sequence similarity to a 16S ribosomal RNA sequence of Bacteroides finegoldii deposited at DSM under number DSM 34013; and/or (d) a bacterial strain having a 16S ribosomal RNA sequence displaying at least 97.0% sequence similarity to a 16S ribosomal RNA sequence of Bacteroides vulgatus deposited at DSM under number DSM 34030. In some embodiments of any of the embodiments disclosed herein, (a)(i) the 16S ribosomal RNA sequence of Barnesiella intestinihominis deposited at DSM under number DSM 34032 comprises SEQ ID NO:1; (ii) the 16S ribosomal RNA sequence displaying at least 97.0% sequence similarity to a 16S ribosomal RNA sequence of Barnesiella intestinihominis deposited at DSM under number DSM 34012 comprises SEQ ID NO:2; (b) the 16S ribosomal RNA sequence of Alistipes onderdonkii deposited at DSM under number DSM 34033 comprises SEQ ID NO:3; (c) the 16S ribosomal RNA sequence of Bacteroides finegoldii deposited at DSM under number DSM 34013 comprises SEQ ID NO:4; (d) the 16S ribosomal RNA sequence displaying at least 97.0% sequence similarity to a 16S ribosomal RNA sequence of Bacteroides vulgatus deposited at DSM under number DSM 34030 comprises SEQ ID NO:5; and/or (c) the 16S ribosomal RNA sequence of an Oscillibacter sp. deposited at the German Collection of Microorganisms and Cell Cultures (DSM) under number DSM 34011 comprises SEQ ID NO:6. In some embodiments of any of the embodiments disclosed herein, the composition comprises (a)(i) the Barnesiella intestinihominis strain deposited at DSM under number DSM 34032 or a live strain having all of the identifying characteristics of the Barnesiella intestinihominis strain deposited at DSM under number DSM 34032; and/or (ii) the Barnesiella intestinihominis strain deposited at DSM under number DSM 34012 or a live strain having all of the identifying characteristics of the B. intestinihominis strain deposited at DSM under number DSM 34012; (b) the Alistipes onderdonkii strain deposited at DSM under number DSM 34033 or a live strain having all of the identifying characteristics of the Alistipes onderdonkii strain deposited at DSM under number DSM 34033; (c) the Bacteroides finegoldii strain deposited at DSM under number DSM 34013 or a live strain having all of the identifying characteristics of the Bacteroides finegoldii strain deposited at DSM under number DSM 34013; (d) the Bacteroides vulgatus strain deposited at DSM under number DSM 34030 or a live strain having all of the identifying characteristics of the Bacteroides vulgatus strain deposited at DSM under number DSM 34030; and/or (c) the Oscillibacter sp. deposited at DSM under number DSM 34011 or a live strain having all of the identifying characteristics of the Oscillibacter sp. deposited at DSM under number DSM 34011 either (A) alone; and/or (B) in combination with a culture supernatant derived from one or more of these strains. In some embodiments of any of the embodiments disclosed herein, the composition is lyophilized or freeze dried. In some embodiments of any of the embodiments disclosed herein, the composition is encapsulated or coated. In some embodiments, the coating is an enteric coating. In some embodiments of any of the embodiments disclosed herein, the composition is a food product, food ingredient, dietary supplement, or medicament. In some embodiments of any of the embodiments disclosed herein, at least about 1×10⁴ CFU/g composition to at least about 1×10¹² CFU/g composition of bacteria is present in the composition. In some embodiments of any of the embodiments disclosed herein, the composition is a probiotic. In some embodiments of any of the embodiments disclosed herein, the composition has been pasteurized or heat treated. In some embodiments of any of the embodiments disclosed herein, the composition is a pharmaceutical composition and further comprises at least one pharmaceutically acceptable carrier and/or excipient. In some embodiments of any of the embodiments disclosed herein, the composition further comprises one or more additional probiotic microorganisms and/or a prebiotic. In some embodiments, at least one of the one or more additional probiotic microorganisms is listed on Table 1.

In other aspects, provided herein is a composition comprising isolated bacterial extracellular vesicles (EVs) derived from at least one or more of (a) a biologically pure strain of Barnesiella intestinihominis; (b) a biologically pure strain of Alistipes onderdonkii; (c) a biologically pure strain of Bacteroides finegoldii; (d) a biologically pure strain of Bacteroides vulgatus; and/or (e) a bacterial strain having a 16S ribosomal RNA sequence displaying at least 97.0% sequence similarity to a 16S ribosomal RNA sequence of an Oscillibacter sp. deposited at the German Collection of Microorganisms and Cell Cultures (DSM) under number DSM 34011. In some embodiments, the composition further comprises one or more bacteria from (a), (b), (c), (d), and/or (c). In some embodiments of any of the embodiments disclosed herein, the composition comprises (a)(i) EVs derived from a bacterial strain having a 16S ribosomal RNA sequence displaying at least 97.0% sequence similarity to a 16S ribosomal RNA sequence of Barnesiella intestinihominis deposited at DSM under number DSM 34032; and/or (ii) EVs derived from a bacterial strain having a 16S ribosomal RNA sequence displaying at least 97.0% sequence similarity to a 16S ribosomal RNA sequence of Barnesiella intestinihominis deposited at DSM under number DSM 34012; (b) EVs derived from a bacterial strain having a 16S ribosomal RNA sequence displaying at least 97.0% sequence similarity to a 16S ribosomal RNA sequence of Alistipes onderdonkii deposited at DSM under number DSM 34033; (c) EVs derived from a bacterial strain having a 16S ribosomal RNA sequence displaying at least 97.0% sequence similarity to a 16S ribosomal RNA sequence of Bacteroides finegoldii deposited at DSM under number DSM 34013; and/or (d) EVs derived from a bacterial strain having a 16S ribosomal RNA sequence displaying at least 97.0% sequence similarity to a 16S ribosomal RNA sequence of Bacteroides vulgatus deposited at DSM under number DSM 34030. In some embodiments of any of the embodiments disclosed herein, the composition comprises (a)(i) the 16S ribosomal RNA sequence of Barnesiella intestinihominis deposited at DSM under number DSM 34032 comprises SEQ ID NO:1; (ii) the 16S ribosomal RNA sequence displaying at least 97.0% sequence similarity to a 16S ribosomal RNA sequence of Barnesiella intestinihominis deposited at DSM under number DSM 34012 comprises SEQ ID NO:2; (b) the 16S ribosomal RNA sequence of Alistipes onderdonkii deposited at DSM under number DSM 34033 comprises SEQ ID NO:3; (c) the 16S ribosomal RNA sequence of Bacteroides finegoldii deposited at DSM under number DSM 34013 comprises SEQ ID NO:4; (d) the 16S ribosomal RNA sequence displaying at least 97.0% sequence similarity to a 16S ribosomal RNA sequence of Bacteroides vulgatus deposited at DSM under number DSM 34030 comprises SEQ ID NO:5; and/or (c) the 16S ribosomal RNA sequence of an Oscillibacter sp. deposited at the German Collection of Microorganisms and Cell Cultures (DSM) under number DSM 34011 comprises SEQ ID NO:6. In some embodiments of any of the embodiments disclosed herein, the composition comprises (a)(i) EVs derived from the Barnesiella intestinihominis strain deposited at DSM under number DSM 34032 or a live strain having all of the identifying characteristics of the Barnesiella intestinihominis strain deposited at DSM under number DSM 34032; and/or (ii) EVs derived from the Barnesiella intestinihominis strain deposited at DSM under number DSM 34012 or a live strain having all of the identifying characteristics of the B. intestinihominis strain deposited at DSM under number DSM 34012; (b) EVs derived from the Alistipes onderdonkii strain deposited at DSM under number DSM 34033 or a live strain having all of the identifying characteristics of the Alistipes onderdonkii strain deposited at DSM under number DSM 34033; (c) EVs derived from the Bacteroides finegoldii strain deposited at DSM under number DSM 34013 or a live strain having all of the identifying characteristics of the Bacteroides finegoldii strain deposited at DSM under number DSM 34013; (d) EVs derived from the Bacteroides vulgatus strain deposited at DSM under number DSM 34030 or a live strain having all of the identifying characteristics of the Bacteroides vulgatus strain deposited at DSM under number DSM 34030; and/or (c) EVs derived from the Oscillibacter sp. deposited at DSM under number DSM 34011 or a live strain having all of the identifying characteristics of the Oscillibacter sp. deposited at DSM under number DSM 34011. In some embodiments of any of the embodiments disclosed herein, the composition is lyophilized or freeze dried. In some embodiments of any of the embodiments disclosed herein, the composition is encapsulated or coated. In some embodiments, the coating is an enteric coating. In some embodiments of any of the embodiments disclosed herein, the composition is a food product, food ingredient, dietary supplement, or medicament. In some embodiments of any of the embodiments disclosed herein, at least about 1×10⁴ CFU/g composition to at least about 1×10¹² CFU/g composition of bacteria is present in the composition. In some embodiments of any of the embodiments disclosed herein, the composition is a probiotic. In some embodiments of any of the embodiments disclosed herein, the composition has been pasteurized or heat treated. In some embodiments of any of the embodiments disclosed herein, the composition is a pharmaceutical composition and further comprises at least one pharmaceutically acceptable carrier and/or excipient. In some embodiments of any of the embodiments disclosed herein, the composition further comprises one or more additional probiotic microorganisms and/or a prebiotic. In some embodiments, at least one of the one or more additional probiotic microorganisms is listed on Table 1.

In still further aspects, provided herein is a tablet, prolonged-release capsule, prolonged-release granule, powder, sachet, or gummy comprising any of the probiotic compositions disclosed herein.

In another aspect, provided herein is a kit comprising (a)(i) any of the probiotic compositions disclosed herein; or (ii) any of the tablet, prolonged-release capsule, prolonged-release granule, powder, sachet, or gummy compositions disclosed herein and b) written instructions for administration to a subject.

In yet additional aspects, provided herein is a method for treating and/or preventing one or more obesity related disorders in a subject in need thereof, comprising administering a therapeutically effective amount of any of the probiotic compositions disclosed herein or any of the tablet, prolonged-release capsule, prolonged-release granule, powder, sachet, or gummy compositions disclosed herein to the subject. In some embodiments, the obesity related disorder is one or more disorders selected from the group consisting of obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease. In some embodiments, cardiovascular disease is one or more of coronary heart disease, cardiomyopathy, ischemic heart disease, heart failure, peripheral arterial disease, hypertension, inflammatory heart disease, valvular heart disease and/or aneurysm. In some embodiments of any of the embodiments disclosed herein, the method results in increased production of one or more of agmatine, carnosine, Ile-Pro-Pro and Val-Pro-Pro bioactive tripeptides, octanoic acid, short chain fatty acids, and/or branched-chain keto acids in the subject.

In another aspect, provided herein is a method for treating and/or preventing viral infection in a subject in need thereof, comprising administering a therapeutically effective amount of any of the probiotic compositions disclosed herein or any of the tablet, prolonged-release capsule, prolonged-release granule, powder, sachet, or gummy compositions disclosed herein to the subject. In some embodiments, the method results in increased production of penciclovir in the subject.

In one aspect, provided herein is a composition for use in the prevention and/or treatment of one or more obesity-related disorders in a subject in need thereof, comprising any of the probiotic compositions disclosed herein or any of the tablet, prolonged-release capsule, prolonged-release granule, powder, sachet, or gummy compositions disclosed herein. In some embodiments, the obesity related disorder is one or more disorders selected from the group consisting of obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease. In some embodiments of any of the embodiments disclosed herein, cardiovascular disease is one or more of coronary heart disease, cardiomyopathy, ischemic heart disease, heart failure, peripheral arterial disease, hypertension, inflammatory heart disease, valvular heart disease and/or aneurysm. In some embodiments of any of the embodiments disclosed herein, the composition results in increased production of one or more of agmatine, carnosine, Ile-Pro-Pro and Val-Pro-Pro bioactive tripeptides, octanoic acid, short chain fatty acids, and/or branched-chain keto acids in the subject.

In another aspect, provided herein is a composition for use in the prevention and/or treatment of viral infection in a subject in need thereof, comprising any of the probiotic compositions disclosed herein or any of the tablet, prolonged-release capsule, prolonged-release granule, powder, sachet, or gummy compositions disclosed herein. In some embodiments, the composition results in increased production of penciclovir in the subject.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts graphs showing the abundance of select probiotic candidates in study groups (lean and obese) at visit 1 (V1) and visit 3 (V3).

FIG. 2 depicts a bar graph showing sample comparison of CE-TOFMS relative peak areas for agmatine for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 3 depicts a bar graph showing sample comparison of CE-TOFMS relative peak areas for carnosine for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 4 depicts a bar graph showing sample comparison of CE-TOFMS relative peak areas for penciclovir for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 5 depicts a bar graph showing sample comparison of CE-TOFMS relative peak areas for the tripeptide Val-Pro-Pro for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 6 depicts a bar graph showing sample comparison of CE-TOFMS relative peak areas for the tripeptide Ile-Pro-Pro for select probiotic candidates. Candidates tested were Barnesiella intestinihominis DSM34032, Barnesiella intestinihominis DSM34012, Alistipes onderdonkii DSM34033, Bacteroides finegoldii DSM34013, and Bacteroides vulgatus DSM34030.

FIG. 7 depicts a bar graph showing sample comparison of CE-TOFMS relative peak areas for octanoic acid for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 8 depicts a bar graph showing sample comparison of succinic acid molar concentration (in mM) by NMR for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 9 depicts a bar graph showing sample comparison of short chain fatty acid (formic, acetic, and propionic acid) molar concentration (in mM) by NMR for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 10A depicts a bar graph showing sample comparison of CE-TOFMS relative peak areas for the branched-chain keto acid (BCKA) 2-oxo-isovaleric acid for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030. FIG. 10B depicts a bar graph showing sample comparison of CE-TOFMS relative peak areas for the BCKA 3-methyl-2-oxo-valeric acid for select probiotic candidates. Candidates tested were Barnesiella intestinihominis DSM34032, Barnesiella intestinihominis DSM34012, Alistipes onderdonkii DSM34033, Bacteroides finegoldii DSM34013, Phocaeicola vulgatus/Bacteroides vulgatus DSM34030, and Oscillibacter sp. DSM34011.

FIG. 11 depicts a bar graph showing sample comparison of CE-TOFMS relative peak areas for 4-Amino-butyric acid (GABA) for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 12 depicts a bar graph showing sample comparison of CE-TOFMS relative peak areas for glutathione for select probiotic candidates. Candidates tested were Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030.

FIG. 13 depicts a phylogenetic tree of Oscillibacter and closely related species constructed based on the alignment of 100 single-copy core genes using RAXML with bootstrap values for 100 iterations.

### DETAILED DESCRIPTION

Many previous studies have shown that probiotic bacteria, for example from the genera Lactobacillus and Bifidobacterium, support the growth of beneficial gut bacteria colonies but it also seems that certain beneficial probiotic strains can also alter host metabolism pathways for the better. Microbial organisms produce bioactive substances that influence carbohydrate and lipid metabolism and modulate both intestinal and systemic inflammatory processes. Thus, there has been increasing interest in identifying nutritional supplements and probiotic foods that are effective for the control of obesity and obesity related disorders.

The inventors of the instant application have surprisingly found that microorganisms outside of the commonly used probiotics Lactobacillus and Bifidobacterium can successfully alter gut metabolism and ameliorate conditions associated with obesity. These beneficial microorganisms were found to be both enriched in the digestive systems of healthy people of normal weight and were deficient in individuals suffering one or more obesity related disorders. Supplementation of one or more of the beneficial microorganisms to the diets of mice modeling human obesity resulted in substantial improvement on one or more metrics relevant to negative conditions associated with obesity.

### I. Definitions

As used herein, “microorganism” or “microbe” refers to a bacterium, a fungus, a virus, a protozoan, and other microbes or microscopic organisms.

As used here in the term “probiotic” refers to a composition for consumption by humans and/or animals (i.e. as an or as a component of animal feed) that contains viable (i.e. live) microorganisms, i.e. microorganisms that are capable of living and reproducing that, when administered in adequate amounts, confer a health benefit on a subject (see Hill et al. 2014 Nature Revs Gastro & Hep 11, 506-514, incorporated by reference herein in its entirety). A probiotic may comprise one or more (such as any of 1, 2, 3, 4, 5, 6, 7, 8, 9, 10 or more) of any of the microbial strains described herein. Probiotics are distinguished from bacterial compositions that have been killed, for example, by pasteurization or heat treatment. Administration of non-viable bacterial compositions for the treatment of one or more metabolic disorders is also contemplated in certain embodiments of the methods disclosed herein.

A bacterial “strain” as used herein refers to a bacterium which remains genetically unchanged when grown or multiplied. The multiplicity of identical bacteria is included.

By “at least one strain,” is meant a single strain but also mixtures of strains comprising at least two strains of microorganisms. By “a mixture of at least two strains,” is meant a mixture of two, three, four, five, six or even more strains. In some embodiments of a mixture of strains, the proportions can vary from 1% to 99%. When a mixture comprises more than two strains, the strains can be present in substantially equal proportions in the mixture or in different proportions.

For purposes of this disclosure, a “biologically pure strain” means a strain containing no other bacterial strains in quantities sufficient to interfere with replication of the strain or to be detectable by normal bacteriological techniques. “Isolated” when used in connection with the organisms and cultures described herein includes not only a biologically pure strain, but also any culture of organisms which is grown or maintained other than as it is found in nature. In some embodiments, the strains are mutants, variants, or derivatives of strains Barnesiella intestinihominis, Alistipes onderdonkii, Bacteroides finegoldii, Bacteroides vulgatus and/or an Oscillibacter sp. displaying at least 97.0% sequence similarity to a 16S ribosomal RNA sequence of an Oscillibacter sp. deposited at the German Collection of Microorganisms and Cell Cultures (DSM) under number DSM 34011 that also provide benefits comparable to that provided by Barnesiella intestinihominis, Alistipes onderdonkii, Bacteroides finegoldii, Bacteroides vulgatus and/or an Oscillibacter sp. displaying at least 97.0% sequence similarity to a 16S ribosomal RNA sequence of an Oscillibacter sp. deposited at the German Collection of Microorganisms and Cell Cultures (DSM) under number DSM 34011. In some embodiments, the strains are strains having all of the identifying characteristics of Barnesiella intestinihominis, Alistipes onderdonkii, Bacteroides finegoldii, Bacteroides vulgatus and/or an Oscillibacter sp. displaying at least 97.0% sequence similarity to a 16S ribosomal RNA sequence of an Oscillibacter sp. deposited at the German Collection of Microorganisms and Cell Cultures (DSM) under number DSM 34011. Further, each individual strain (Barnesiella intestinihominis, Alistipes onderdonkii, Bacteroides finegoldii, Bacteroides vulgatus and/or an Oscillibacter sp. displaying at least 97.0 % sequence similarity to a 16S ribosomal RNA sequence of an Oscillibacter sp. deposited at the German Collection of Microorganisms and Cell Cultures (DSM) under number DSM 34011) or any combination of these strains can also provide one or more of the benefits described herein. It will also be clear that addition of other microbial strains, carriers, additives, enzymes, yeast, or the like will also provide one or more benefits or improvement of one or more metabolic conditions in a subject and will not constitute a substantially different bacterial strain.

The term “16S rRNA” or “16S ribosomal RNA” means the rRNA constituting the small subunit of prokaryotic ribosomes. In bacteria, this sequence can be used to identify and characterize operational taxonomic units.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

As used herein, the term “subject” or “patient” is meant a mammal (e.g., a human). In some embodiments, a subject is suffering from a relevant disease, disorder or condition such as, without limitation, one or more metabolic disorders, for example, obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, “prevent,” “preventing,” “prevention” and grammatical variations thereof refers to a method of partially or completely delaying or precluding the onset or recurrence of a disorder or condition (such as one or more metabolic disorders, for example, obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease) and/or one or more of its attendant symptoms or barring a subject from acquiring or reacquiring a disorder or condition or reducing a subject's risk of acquiring or reacquiring a disorder or condition or one or more of its attendant symptoms.

As used herein, “cardiovascular disease” or “heart disease” is a term used to describe a range of diseases or events that affect the heart and/or vasculature. Types of heart disease include, but are not limited to, coronary heart disease, cardiomyopathy, ischemic heart disease, hypertension, peripheral arterial disease, heart failure, inflammatory heart disease, valvular heart disease and aneurysm. Heart disease can be assessed using clinical parameters and/or assessments known to those skilled in the art of diagnosing and/or treating the same, for example, physical examinations, detection of signs and symptoms of cardiovascular disease, electrocardiogram, echocardiogram, chest X-ray, blood tests to detect cardiac biomarkers, etc. Biomarkers typically used in the clinical setting include, but are not limited to, cardiac troponins (C. T. and I), CK and CK-MB, and myoglobin.

As used herein, the term “reducing” in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%.

As used herein, the term “increasing” in relation to a particular trait, characteristic, feature, biological process, or phenomena refers to an increase in the particular trait, characteristic, feature, biological process, or phenomena. The trait, characteristic, feature, biological process, or phenomena can be increased by 5%, 10%, 15%, 20%, 25%, 30%, 35%, 40%, 45%, 50%, 55%, 60%, 65%, 70%, 75%, 80%, 85%, 90%, 95%, 100% 105%, 110%, 115%, 120%, 125%, 130%, 135%, 140%, 145%, 150%, 160%, 170%, 180%, 190%, 200%, 250%, 300%, 350%, 400%, 450%, 500%, or greater than 500%.

As used herein “administer” or “administering” is meant the action of introducing one or more compositions comprising one or more microbial strain, to a subject, such as by feeding or consuming orally. The composition containing one or more microbial strains can also be administered in one or more doses.

As used herein, “effective amount” means a quantity of a composition containing one or more microbial strains to improve one or more metrics in subject. Improvement in one or more metrics of an subject (such as, without limitation, any of treating and/or preventing obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease) can be measured as described herein or by other methods known in the art.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains. Other definitions of terms may appear throughout the specification.

### II. Compositions

### A. Strains

The beneficial microbial-containing compositions disclosed herein can be used as supplements, food additives, and therapeutics for administration to subjects when under periods of physiologic stress (disease state, metabolic state, etc.) or as a part of a daily nutritional regimen to prevent disease and facilitate healthy gut metabolism. Probiotics is another term that can be used for these compositions which contain viable microorganisms. The term “viable microorganism” means a microorganism which is metabolically active or able to differentiate. In some embodiments, the beneficial microbial-containing compositions disclosed herein include both viable probiotic products and/or, in particular embodiments, compositions that include non-viable bacteria (such as heat-treated or pasteurized compositions).

The strains provided herein include two biologically pure strains of Barnesiella intestinihominis, a biologically pure strain of Alistipes onderdonkii; a biologically pure strain of Bacteroides finegoldii; a biologically pure strain of Bacteroides vulgatus; and a biologically pure bacterial strain having a 16S ribosomal RNA sequence displaying at least 97.0% sequence similarity to a 16S ribosomal RNA sequence of an Oscillibacter sp. deposited at the German Collection of Microorganisms and Cell Cultures (DSM) under number DSM 34011. As used herein, “Bacteroides vulgatus” can also refer interchangeably to “Phocaeicola vulgatus” per recent reclassification of this species (Oren & Garrity, 2020, International Journal of Systematic and Evolutionary Microbiology, 70 (5): 2960-66, incorporated by reference herein).

The two B. intestinihominis strains, the A. onderdonkii strain, the B. finegoldii strain, the B. vulgatus strain, and the Oscillibacter sp. strain were deposited on Sep. 8, 2021 at the German Collection of Microorganisms and Cell Cultures GmbH (DSM), Inhoffenstraβe 7B, 38124 Braunschweig, GERMANY and given accession numbers DSM 34032, DSM 34012, DSM 34033, DSM 34013, and DSM 34011, respectively. The deposits were made under the provisions of the Budapest Treaty on the International Recognition of the Deposit of Microorganisms for the Purposes of Patent Procedure. One or more strain provided herein can be used as a probiotic in one non-limiting embodiment.

The microbial-containing compositions (such as probiotic compositions) can include those that contain one or more strains (such as any of about 1, 2, 3, 4, 5, 6, 7, or 8 or more strains) of Barnesiella intestinihominis (such as B. intestinihominis strain DSM 34032 and/or B. intestinihominis strain DSM 34012). Barnesiella intestinihominis is a Gram-negative, anaerobic and non-spore-forming bacterium from the genus of Barnesiella. The beneficial microbial-containing compositions disclosed herein can further include those that contain one or more strains (such as any of about 1, 2, 3, 4, 5, 6, 7, or 8 or more strains) of A. onderdonkii, B. finegoldii, B. vulgatus, and/or Oscillibacter sp.; and/or one or more strains (such as any of about 1, 2, 3, 4, 5, 6, 7, or 8 or more strains) of E. eligens, I. massiliensis, P. copri, and/or Akkermansia sp. (such as those disclosed in International Patent Application Publication No. WO2021203083, incorporated by reference herein); and/or one or more strains (such as any of about 1, 2, 3, 4, 5, 6, 7, or 8 or more strains) disclosed herein in Table 1 of Example 1.

The microbial-containing compositions (such as probiotic compositions) can include those that contain one or more strains (such as any of about 1, 2, 3, 4, 5, 6, 7, or 8 or more strains) of Alistipes onderdonkii (such as A. onderdonkii strain DSM 34033). A. onderdonkii is a Gram-negative, rod-shaped and anaerobic bacterium from the genus of Alistipes. The beneficial microbial-containing compositions disclosed herein can further include one or more strains (such as any of about 1, 2, 3, 4, 5, 6, 7, or 8 or more strains) of B. intestinihominis, B. finegoldii, B. vulgatus, and/or Oscillibacter sp.; and/or one or more strains (such as any of about 1, 2, 3, 4, 5, 6, 7, or 8 or more strains) of E. eligens, I. massiliensis, P. copri, and/or Akkermansia sp. (such as those disclosed in International Patent Application Publication No. WO2021203083, incorporated by reference herein); and/or one or more strains (such as any of about 1, 2, 3, 4, 5, 6, 7, or 8 or more strains) disclosed herein in Table 1 of Example 1.

The microbial-containing compositions (such as probiotic compositions) can include those that contain one or more strains (such as any of about 1, 2, 3, 4, 5, 6, 7, or 8 or more strains) of Bacteroides finegoldii (such as B. finegoldii strain DSM 34013). B. finegoldii is a strictly anaerobic, Gram-negative rod bacteria commonly found in the gut. The beneficial microbial-containing compositions disclosed herein can further include those that contain one or more strains (such as any of about 1, 2, 3, 4, 5, 6, 7, or 8 or more strains) B. intestinihominis, A. onderdonkii, B. vulgatus, and/or Oscillibacter sp.; and/or one or more strains (such as any of about 1, 2, 3, 4, 5, 6, 7, or 8 or more strains) of E. eligens, I. massiliensis, P. copri, and/or Akkermansia sp. (such as those disclosed in International Patent Application Publication No. WO2021203083, incorporated by reference herein); and/or one or more strains (such as any of about 1, 2, 3, 4, 5, 6, 7, or 8 or more strains) disclosed herein in Table 1 of Example 1.

The microbial-containing compositions (such as probiotic compositions) can include those that contain one or more strains (such as any of about 1, 2, 3, 4, 5, 6, 7, or 8 or more strains) of Bacteroides vulgatus (such as B. vulgatus strain DSM 34030). B. vulgatus is a Gram-negative, obligate anaerobic bacteria. The beneficial microbial-containing compositions disclosed herein can further include those that contain one or more strains (such as any of about 1, 2, 3, 4, 5, 6, 7, or 8 or more strains) B. intestinihominis, A. onderdonkii, B. finegoldii, and/or Oscillibacter sp.; and/or one or more strains (such as any of about 1, 2, 3, 4, 5, 6, 7, or 8 or more strains) of E. eligens, I. massiliensis, P. copri, and/or Akkermansia sp. (such as those disclosed in International Patent Application Publication No. WO2021203083, incorporated by reference herein); and/or one or more strains (such as any of about 1, 2, 3, 4, 5, 6, 7, or 8 or more strains) disclosed herein in Table 1 of Example 1.

The microbial-containing compositions (such as probiotic compositions) can include those that contain one or more strains (such as any of about 1, 2, 3, 4, 5, 6, 7, or 8 or more strains) of an Oscillibacter sp., where the Oscillibacter sp. has a 16S ribosomal RNA sequence displaying at least 97.0% sequence similarity to a 16S ribosomal RNA sequence of an Oscillibacter sp. deposited at the German Collection of Microorganisms and Cell Cultures (DSM) under number DSM 34011. The beneficial microbial-containing compositions disclosed herein can further include those that contain one or more strains (such as any of about 1, 2, 3, 4, 5, 6, 7, or 8 or more strains) B. intestinihominis, A. onderdonkii, B. finegoldii, and/or B. vulgatus; and/or one or more strains (such as any of about 1, 2, 3, 4, 5, 6, 7, or 8 or more strains) of E. eligens, I. massiliensis, P. copri, and/or Akkermansia sp. (such as those disclosed in International Patent Application Publication No. WO2021203083, incorporated by reference herein); and/or one or more strains (such as any of about 1, 2, 3, 4, 5, 6, 7, or 8 or more strains) disclosed herein in Table 1 of Example 1.

The microbial-containing compositions (such as probiotic compositions) disclosed herein can include one or more Barnesiella intestinihominis strain (such as B. intestinihominis strain DSM 34032 and/or B. intestinihominis strain DSM 34012) having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO: 1 and/or SEQ ID NO:2. The beneficial microbial-containing compositions (such as probiotic compositions) can include one or more A. onderdonkii strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO:3. The beneficial microbial-containing compositions (such as probiotic compositions) can include one or more B. finegoldii strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO:4. The beneficial microbial-containing compositions (such as probiotic compositions) can include one or more B. vulgatus strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO:5. The beneficial microbial-containing compositions (such as probiotic compositions) can include one or more Oscillibacter sp. strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO:6.

The microbial-containing compositions (such as probiotic compositions) disclosed herein can include one or more Barnesiella intestinihominis strain (such as B. intestinihominis strain DSM 34032 and/or B. intestinihominis strain DSM 34012), one or more Alistipes onderdonkii; strain (such as A. onderdonkii strain DSM 34033), one or more Bacteroides finegoldii strain (such as B. finegoldii strain DSM 34013), one or more Bacteroides vulgatus strain (such as B. vulgatus strain DSM 34030), and/or one or more Oscillibacter sp. strain (such as Oscillibacter sp. strain DSM 34011). The compositions can include the actual bacteria (viable or non-viable) from these strains and/or one or more culture supernatants derived from the culturing of these strains (individually or in co-culture).

The microbial-containing compositions (such as probiotic compositions) can include those that contain one or more strains (such as any of about 1, 2, 3, 4, 5, 6, 7, or 8 or more strains) of disclosed herein in Table 1 of Example 1.

### B. Formulations

Generally, the microbial-containing compositions (such as probiotic compositions) disclosed herein comprise bacteria, such as one or more bacterial strains. In some embodiments of the invention, the composition is formulated in freeze-dried or lyophilized form. For example, the microbial-containing compositions can comprise granules or gelatin capsules, for example hard gelatin capsules, comprising a bacterial strain disclosed herein.

In some embodiments, the microbial-containing compositions disclosed herein comprise lyophilized bacteria. Lyophilization of bacteria is a well-established procedure in the art. Alternatively, the microbial-containing compositions can comprise a live, active bacterial culture.

In some embodiments, any of the microbial-containing compositions disclosed herein is encapsulated to enable delivery of the bacterial strain to the intestine. Encapsulation protects the composition from degradation until delivery at the target location through, for example, rapturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule.

The microbial-containing compositions disclosed herein can be administered orally and may be in the form of a tablet, capsule or powder. Other ingredients (such as vitamin C or minerals, for example), may be included as oxygen scavengers and prebiotic substrates to improve the delivery and/or partial or total colonization and survival in vivo. Alternatively, the microbial-containing compositions (such as probiotic compositions) disclosed herein can be administered orally as a food or nutritional product, such as milk or whey based fermented dairy product, or as a pharmaceutical product.

The microbial-containing compositions disclosed herein can be formulated as a probiotic. Alternatively, the microbial-containing compositions disclosed herein can be formulated as a non-viable bacterial compositions, such as a pasteurized or heat-treated bacterial composition.

A microbial-containing composition disclosed herein includes a therapeutically effective amount of a bacterial strain disclosed herein. A therapeutically effective amount of a bacterial strain is sufficient to exert a beneficial effect upon a patient A therapeutically effective amount of a bacterial strain may be sufficient to result in delivery to and/or partial or total colonization of the subject's intestine.

A suitable daily dose of the bacteria, for example for an adult human, may be from about 1×10³ to about 1×10¹¹ colony forming units (CPU); for example, from about 1×10⁷ to about 1×10¹⁰ CPU; in another example from about 1×10⁶ to about 1×10¹⁰ GPU; in another example from about 1×10⁷ to about 1×10¹¹ CPU; in another example from about 1×10⁸ to about 1×10¹⁰ CPU; in another example from about 1×10⁸ to about 1×10¹¹ CPU. In certain embodiments, the dose of the bacteria is at least 109 cells per day, such as at least 10¹⁰, at least 10¹¹ or at least 10¹² cells per day.

In certain embodiments, the microbial-containing composition contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

In certain embodiments, the amount of the bacterial strain is from about 1×10³ to about 1×10¹¹ colony forming units per gram with respect to a weight of the composition.

In certain embodiments, any of the microbial-containing compositions disclosed herein is administered at a dose of between 500 mg and 1000 mg, between 600 mg and 900 mg, between 700 mg and 800 mg, between 500 mg and 750 mg or between 750 mg and 1000 mg. In certain embodiments, the lyophilized bacteria in any of the microbial-containing compositions disclosed herein is administered at a dose of between 500 mg and 100 mg, between 600 mg and 900 mg, between 700 mg and 800 mg, between 500 mg and 750 mg or between 750 mg and 1000 mg.

Typically, a probiotic, is optionally combined with at least one suitable prebiotic compound. A prebiotic compound is usually a non-digestible carbohydrate such as an oligo-or polysaccharide, or a sugar alcohol, which is not degraded or absorbed in the upper digestive tract Known prebiotics include commercial products such as inulin and transgalacto-oligosaccharides.

In certain embodiments, a probiotic composition disclosed herein is formulated to include a prebiotic compound in an amount of from about 1 to about 30% by weight respect to the total weight composition, (e.g. from 5 to 20% by weight). Carbohydrates may be selected from the group consisting of: fructo-oligosaccharides (or FOS), short-chain fructo-ol igosaccharides, inulin, isomalt-oligosaccharides, pectins, xylo-oligosaccharides (or XOS), chitosan-oligosaccharides (or COS), human milk oligosaccharides, beta-glucans, gum arabic modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded. In another embodiment, the prebiotic can include one or more polyphenols (such as a plant polyphenol). In some embodiments, any of the probiotics disclosed herein can be formulated with additional probiotics derived from the genera Lactobacillus and Bifidobacterium (such as B. lactis B420).

The microbial-containing composition disclosed herein can further comprise pharmaceutically acceptable excipients or carriers. Acceptable carriers or diluents for therapeutic use are well known in the pharmaceutical art. Examples of suitable carriers include, without limitation, lactose, starch, glucose, methyl cellulose, magnesium stearate, mannitol, sorbitol and the like. Examples of suitable diluents include, without limitation, ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binders, lubricants, suspending agents, coating agents (such as a gastric-resistant enteric coating agent that does not dissolve or degrade until reaching the small or large intestine), or solubilizing agents. Examples of suitable binders include, without limitation, starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include, without limitation, sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include, without limitation, sodium benzoate, sorbic acid and esters of p-hydroxybenzoic acid. Antioxidants and suspending agents may be also used.

The microbial-containing composition disclosed herein can be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the microbial-containing composition is formulated as a milk-based product. The term “milk-based product,” as used herein, means any liquid or semi-solid milk-or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavored milks, ice cream; milk-containing food such as sweets.

In certain embodiments, the microbial-containing compositions disclosed herein contain a single bacterial strain or species and do not contain any other bacterial strains or species. Such compositions may comprise only de minimis or biologically irrelevant amounts of other bacterial strains or species. Such compositions may be a culture that is substantially free from other species of organism. In certain embodiments, the compositions of the invention consist of 1, 2, 3, 4, 5, 6, 7, 8, 9, 10, 11, 12, 13, 14, 15 or 16 bacterial strains or species. In certain embodiments, the compositions consist of from 1 to 10, such as from 1 to 5 bacterial strains or species.

The microbial-containing composition for use in accordance with the methods disclosed herein may or may not require marketing approval.

In some cases, the lyophilized bacterial strain is reconstituted prior to administration. In some cases, the reconstitution is by use of a diluent described herein.

The microbial-containing compositions disclosed herein can comprise pharmaceutically acceptable excipients, diluents or carriers.

In certain embodiments, provided herein is a pharmaceutical composition comprising: a bacterial strain disclosed herein; and a pharmaceutically acceptable excipient, carrier or diluent; wherein the bacterial strain is in an amount sufficient to treat a disorder when administered to a subject in need thereof; and wherein the disorder is selected from the group consisting of obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilized.

In certain embodiments, the above pharmaceutical composition, wherein when the composition is stored in a sealed container at about 4° C. or about 25° C. and the container is placed in an atmosphere having 50% relative humidity, at least 80%, 70%, 60%, 50%, 40%, 30%, 20%, or 10% of the bacterial strain as measured in colony forming units, remains after a period of at least about 1 month, 3 months, 6 months, 1 year, 1.5 years, 2 years, 2.5 years or 3 years.

The bacterial strains disclosed herein can be cultured using standard microbiology techniques such as those described in the Examples section or that are well known in the art.

In additional embodiments, one or more of the bacterial strains disclosed herein can be formulated as compositions (such as pharmaceutical compositions) comprising bacterial extracellular vesicles (EVs). As used herein, the term “extracellular vesicle” or “EV” or refers to a composition derived from a bacterium that comprises bacterial lipids, and bacterial proteins and/or bacterial nucleic acids and/or carbohydrate moieties contained in a nanoparticle. These EVs may contain 1, 2, 3, 4, 5, 10, or more than 10 different lipid species. EVs may contain 1, 2, 3, 4, 5, 10, or more than 10 different protein species. EVs may contain 1, 2, 3, 4, 5, 10, or more than 10 different nucleic acid species. EVs may contain 1, 2, 3, 4, 5, 10, or more than 10 different carbohydrate species. As used herein, the term “purified EV composition” or “EV composition” refer to a preparation that includes EVs that have been separated from at least one associated substance found in a source material (e.g. separated from at least one other bacterial component) or any material associated with the EVs in any process used to produce the preparation. It also refers to a composition that has been significantly enriched or concentrated. In some embodiments the EVs are concentrated by 2-fold, 3-fold, 4-fold, 5-fold, 10 -fold, 100-fold, 1000-fold, 10,000-fold or more than 10,000-fold.

The EVs described herein can be prepared using any method known in the art. In some embodiments, the EVs are prepared without an EV purification step. For example, in some embodiments, bacteria comprising the EVs described herein are killed using a method that leaves the bacterial EVs intact and the resulting bacterial components, including the EVs, are used in the methods and compositions described herein. In some embodiments, the bacteria are killed using an antibiotic (e.g., using an antibiotic described herein). In some embodiments, the bacteria are killed using UV irradiation. In some embodiments, the EVs described herein are purified from one or more other bacterial components. Methods for purifying EVs from bacteria are known in the art. In some embodiments EVs are prepared from bacterial cultures using methods described in S. Bin Park, et al. PLOS ONE. 6(3): el7629 (2011) or G. Norheim, et al. PLOS ONE. 10(9): c0134353 (2015), cach of which is hereby incorporated by reference in its entirety. In some embodiments, the bacteria are cultured to high optical density and then centrifuged to pellet bacteria (e.g., at 10,000× g for 30 min at 4° C.). In some embodiments, the culture supernatants are then passed through filter to exclude intact bacterial cells (e.g., a 0.22 μm filter). In some embodiments, filtered supernatants are centrifuged to pellet bacterial EVs (e.g., at 100,000-150,000× g for 1-3 hours at 4° C.). In some embodiments, the EVs are further purified by resuspending the resulting EV pellets (e.g., in PBS), and applying the resuspended EVs to sucrose gradient (e.g., a 30-60% discontinuous sucrose gradient), followed by centrifugation (e.g., at 200,000× g for 20 hours at 4° C.). EV bands can be collected, washed with (e.g., with PBS), and centrifuged to pellet the EVs (e.g., at 150,000× g for 3 hours at 4° C.). The purified EVs can be stored, for example, at −80° C. until use. In some embodiments, the EVs are further purified by treatment with DNase and/or proteinase K.

For example, in some embodiments, cultures of bacteria disclosed herein can be centrifuged at 11,000× g for 20-40 min at 4° C. to pellet bacteria. Culture supernatants may be passed through a 0.22 μm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000× g for 20-40 min at 4° C. The resulting pellets contain bacterial EVs and other debris.

Using ultracentrifugation, filtered supernatants can be centrifuged at 100,000-200,000× g for 1-16 hours at 4° C. The pellet of this centrifugation contains bacterial EVs and other debris. In some embodiments, using a filtration technique, such as through the use of an Amicon Ultra spin filter or by tangential flow filtration, supernatants can be filtered so as to retain species of molecular weight >50 or 100 kDa.

Alternatively, EVs can be obtained from bacterial cultures continuously during growth, or at selected time points during growth, by connecting a bioreactor to an alternating tangential flow (ATF) system (e.g., XCell ATF from Repligen). The ATF system retains intact cells (>0.22 μm) in the bioreactor, and allows smaller components (e.g., EVs, free proteins) to pass through a filter for collection. For example, the system may be configured so that the <0.22 um filtrate is then passed through a second filter of 100 kDa, allowing species such as EVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. EVs collected by this method may be further purified and/or concentrated by ultracentrifugation or filtration as described above for filtered supernatants.

EVs obtained by methods provided herein may be further purified by size-based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000× g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000× g for 3-24 hours at 4° C.

In some embodiments, to confirm sterility and isolation of the EV preparations, EVs are serially diluted onto agar medium used for routine culture of the bacteria being tested and incubated using routine conditions. Non-sterile preparations are passed through a 0.22 μm filter to exclude intact cells. To further increase purity, isolated EVs may be DNase or proteinase K treated.

### III. Methods

### A. Methods for Treating or Preventing Disease

Further provided herein are methods for treating and/or preventing one or more obesity related disorders including obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease in a subject in need thereof, comprising administering to the subject a therapeutically effective amount of any of the microbial-containing and/or EV-containing compositions disclosed herein.

The body mass index (BMI) (calculated as weight in kilograms divided by the square of height in meters) is the most commonly accepted measurement for overweight and/or obesity. In adults, a BMI exceeding 25 is considered overweight, while obesity is defined as a BMI of 30 or more, with a BMI of 35 or more considered as serious co-morbidity and a BMI of 40 or more considered morbid obesity. For the purposes of this invention, “obesity” shall mean a BMI of 30 or more.

One out of every five overweight people is affected by the “metabolic syndrome”. Metabolic syndrome is one of the fastest growing obesity-related health concerns in the United States and is characterized by a cluster of health problems including obesity, hypertension, abnormal lipid levels, and high blood sugar. According to the Centers for Disease Control and Prevention (CDC), the metabolic syndrome affects almost one quarter (22 percent) of the American population—an estimated 47 million people. The assemblage of problems characterized as comprising the metabolic syndrome can increase a patient's risk for developing more serious health problems, such as diabetes, heart disease, and stroke.

Overweight and obese people have an increased incidence of heart disease, and thus fall victim to heart attack, congestive heart failure, sudden cardiac death, angina, and abnormal heart rhythm more often than those that maintain a healthy body mass index. Obesity often increases the risk of heart disease because of its negative effect on blood lipid levels, which increase in obese patients and then, in turn, increase triglyceride levels and decrease high-density lipoprotein—which is also known as HDL. People with an excessive amount of body fat have higher levels of triglycerides and low-density lipoprotein—which is also known as LDL or “bad cholesterol”—as well as lower levels of HDL cholesterol in the blood. This combination creates optimal conditions for developing atherosclerotic heart disease.

Being overweight or obese increases the risk of developing high blood pressure. Hypertension, or high blood pressure, greatly raises the risk of heart attack, stroke, and kidney failure. In fact, blood pressure rises as body weight increases. Losing even 10 pounds can lower blood pressure—and losing weight has the biggest effect on those who are overweight and already have hypertension.

Obesity is associated with the development of diabetes. More than 80 percent of people with type 2 diabetes, the most common form of the disease, are obese or overweight. Type 2 diabetes develops when either there is impaired insulin production by the pancreas in the setting of insulin resistance in the tissues and organs in the body. As obesity diminishes insulin's ability to control blood sugar (glucose), there is an increased risk of developing diabetes because the body begins overproducing insulin to regulate blood sugar levels. Over time, the body is no longer able to keep blood sugar levels in the normal range. Eventually the inability to achieve healthy blood sugar balance results in the development of type 2 diabetes. Furthermore, obesity complicates the management and treatment of type 2 diabetes by increasing insulin resistance and glucose intolerance, which makes drug treatment for the disease less effective. In many cases, a reduction of body weight to a normal range normalizes blood glucose and restores insulin sensitivity.

Childhood obesity is also a major public health problem, particularly in Western countries. Children 2-18 years of age are considered obese if the BMI is greater than the 95th percentile. Despite policies targeted at reducing its prevalence, childhood obesity has more than doubled in children and tripled in adolescents in the past 30 years. As with adults, obesity in childhood causes hypertension, dyslipidemia (abnormal lipid metabolism), chronic inflammation, increased blood clotting tendency, endothelial dysfunction, and hyperinsulinemia. This clustering of cardiovascular disease risk factors has been identified in children as young as 5 years of age.

The methods disclosed herein are directed to the prevention, inhibition and treatment of obesity-related disorders. An “obesity-related disorder” as used herein, includes, but is not limited to, obesity, undesired weight gain, and an over-eating disorder (e.g., binge eating, bulimia, compulsive eating, or a lack of appetite control each of which can optionally lead to undesired weight gain or obesity), metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease. “Obesity” and “obese” as used herein, refers to class I obesity, class II obesity, class III obesity and pre-obesity (e.g., being “over-weight”) as defined by the World Health Organization.

Decreased body fat is expected to provide various primary and/or secondary benefits in a subject (e.g., in a subject diagnosed with a complication associated with obesity, such as an obesity related disorder) such as, for example, an increased insulin responsiveness or decreased glucose intolerance (e.g., in a subject diagnosed with Type II diabetes mellitus); a reduction in elevated blood pressure; a reduction in elevated cholesterol levels and/or LDLs and/or VLDLs; a reduction (or a reduced risk or progression) of cardiovascular disease (including ischemic heart disease, arterial vascular disease, angina, myocardial infarction, and/or stroke), migraines, congestive heart failure, deep vein thrombosis, pulmonary embolism, gall stones, gastroesophageal reflux disease, obstructive sleep apnea, obesity hypoventilation syndrome, asthma, gout, poor mobility, back pain, erectile dysfunction, urinary incontinence, liver injury (e.g., fatty liver disease, liver cirrhosis, alcoholic cirrhosis, endotoxin-mediated liver injury), chronic renal failure, leptin resistance, and elevated resistin levels.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to reduce obesity. In some embodiments, the subject's obesity decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to obese subjects who are not administered one or more of the microbial-containing and/or EV-containing compositions disclosed herein. Reduction in obesity can be measured by any method known in the art, such as reduction in BMI.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to reduce one or more of metabolic syndrome, diabetes (including type II diabetes), insulin resistance, and/or glucose intolerance. In some embodiments, the rate of one or more of metabolic syndrome, diabetes (including type II diabetes), insulin resistance, and/or glucose intolerance decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects diagnosed with one or more of these conditions who are not administered one or more of the microbial-containing and/or EV-containing compositions disclosed herein. Reduction in one or more of metabolic syndrome, diabetes (including type II diabetes), insulin resistance, and/or glucose intolerance can be determined by any means known in the art, such as blood glucose measurement and determination of AlC.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to treat one or more hepatic disorders (including, without limitation, abnormal lipid metabolism, non-alcoholic fatty liver disease, and/or hepatic steatosis). In some embodiments, incidence of the hepatic disorder decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects with hepatic disorders who are not administered one or more of the microbial-containing and/or EV-containing compositions disclosed herein. Reduction in one or more hepatic disorders can be determined by any means known in the art.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to treat leptin resistance and/or reduced resistin levels. In some embodiments, leptin resistance decreases and/or resistin levels increase by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to subjects with leptin resistance and/or reduced resistin levels who are not administered one or more of the microbial-containing and/or EV-containing compositions disclosed herein. Leptin resistance and/or reduced resistin levels can be determined by any means known in the art.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to treat one or more disorders associated with cardiovascular disease (including, without limitation, ischemic heart disease, arterial vascular disease, angina, myocardial infarction, and/or stroke). In some embodiments, incidence of the cardiovascular disease decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects with one or more disorders associated with cardiovascular disease who are not administered one or more of the microbial-containing and/or EV-containing compositions disclosed herein. Reduction in one or more disorders associated with cardiovascular disease can be determined by any means known in the art.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to increase agmatine levels in the gut. Agmatine is a chemical substance which is naturally created from the amino acid arginine. Agmatine, also known as (4-aminobutyl)guanidine, has been shown to exert modulatory action at multiple molecular targets, notably: neurotransmitter systems, ion channels, nitric oxide (NO) synthesis and polyamine metabolism. In some embodiments, agmatine production and/or gut agmatine levels increase by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects who are not administered one or more of the microbial-containing and/or EV-containing compositions disclosed herein.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to increase carnosine levels in the gut. Carnosine (beta-alanyl-L-histidine) is a dipeptide molecule, made up of the amino acids beta-alanine and histidine that has anti-oxidant properties. In some embodiments, carnosine production and/or gut carnosine levels increase by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects who are not administered one or more of the microbial-containing and/or EV-containing compositions disclosed herein.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to increase levels of Ile-Pro-Pro and Val-Pro-Pro bioactive tripeptides in the gut. In some embodiments, Ile-Pro-Pro and Val-Pro-Pro bioactive tripeptide production and/or gut Ile-Pro-Pro and Val-Pro-Pro bioactive tripeptide levels increase by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects who are not administered one or more of the microbial-containing and/or EV-containing compositions disclosed herein.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to increase octanoic acid levels in the gut. In some embodiments, octanoic acid production and/or gut octanoic acid levels increase by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects who are not administered one or more of the microbial-containing and/or EV-containing compositions disclosed herein.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to increase short chain fatty acids (such as, without limitation, formic, acidic, and/or propionic acid) levels in the gut. In some embodiments, short chain fatty acid production and/or gut short chain fatty acid levels increase by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects who are not administered one or more of the microbial-containing and/or EV-containing compositions disclosed herein.

In another embodiment, the disclosure relates to a method comprising administering to a subject an effective amount of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to increase branched-chain keto acids (such as, without limitation, 2-oxo-isovaleric acid and/or 3-methyl-2-oxo-valeric acid) levels in the gut. In some embodiments, short chain fatty acid production and/or gut branched-chain keto acids levels increase by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects who are not administered one or more of the microbial-containing and/or EV-containing compositions disclosed herein.

In further embodiments, the disclosure relates to a method for treating and/or preventing viral infection in a subject in need thereof, comprising administering a therapeutically effective amount of the composition of any of the microbial-containing and/or EV-containing compositions (such as probiotic compositions containing one or more of B. intestinihominis, A. onderdonkii, B. finegoldii, B. vulgatus, or an Oscillibacter sp.) disclosed herein to increase production of penciclovir in the subject. In some embodiments, penciclovir levels increase by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to subjects who are not administered one or more of the microbial-containing and/or EV-containing compositions disclosed herein.

In another embodiment, the disclosure relates to a method comprising administering to a subject an effective amount of any of the microbial-containing and/or EV-containing compositions comprising one or more strains (such as any of about 1, 2, 3, 4, 5, 6, 7, or 8 or more strains) disclosed herein in Table 1 of Example 1 to improve metabolic health. Improving metabolic health, in some non-limiting embodiments, can include improvements in one or more of obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease in the subject.

In still another embodiment, the microbial-containing and/or EV-containing compositions (such as probiotic compositions) disclosed herein administered to the subject comprises one or more Barnesiella intestinihominis strain (such as B. intestinihominis strain DSM 34032 and/or B. intestinihominis strain DSM 34012), having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO: 1and/or SEQ ID NO:2. The beneficial microbial-containing and/or EV-containing compositions (such as probiotic compositions) can include one or more A. onderdonkii strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO:3. The beneficial microbial-containing and/or EV-containing compositions (such as probiotic compositions) can include one or more B. finegoldii strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO:4. The beneficial microbial-containing and/or EV-containing compositions (such as probiotic compositions) can include one or more B. vulgatus strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO:5. The beneficial microbial-containing and/or EV-containing compositions (such as probiotic compositions) can include one or more Oscillibacter sp. strain having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence comprising SEQ ID NO: 6.

In some embodiments, the one or more (such as 1, 2, 3, 4, 5, 6, 7 or 8) B. intestinihominis, A. onderdonkii, B. finegoldii, B. vulgatus and/or Oscillibacter sp. strain(s) is (are) administered to subject at a rate of at least about 1×10⁴ CFU/subject/day to at least about 1×10¹² CFU/subject/day, such as any of about 1×10⁴ CFU/subject/day, 1×10⁵ CFU/subject/day, 1×10⁶ CFU/subject/day, 1×10⁷ CFU/subject/day, 1×10⁸ CFU/subject/day, 1×10⁹ CFU/subject/day, 1×10¹⁰ CFU/subject/day, 1×10¹¹ CFU/subject/day, or 1×10¹² CFU/subject/day, inclusive of all values falling in between these figures.

### B. Administration

Preferably, the microbial-containing and/or EV-containing compositions disclosed herein are to be administered to the gastrointestinal tract in order to enable delivery to and/or partial or total colonization of the intestine with the bacterial strain of the invention. Generally, the compositions of the invention are administered orally, but they may be administered rectally, intranasally, or via buccal or sublingual routes.

In certain embodiments, the microbial-containing and/or EV-containing compositions disclosed herein may be administered as a foam, as a spray or a gel.

In certain embodiments, the microbial-containing and/or EV-containing compositions disclosed herein of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the microbial-containing and/or EV-containing compositions disclosed herein are administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to tire stomach, jejunum and other suitable access ports.

The microbial-containing and/or EV-containing compositions disclosed herein may be administered once, or they may be administered sequentially as part of a treatment regimen. In certain embodiments, the compositions of the invention are to be administered daily.

In certain embodiments of the invention, treatment with the microbial-containing and/or EV-containing compositions disclosed herein according to methods disclosed herein is accompanied by assessment of the subject's gut microbiota. Treatment may be repeated if delivery of and/or partial or total colonization with the strain of the invention is not achieved such that efficacy is not observed, or treatment may be ceased if delivery and/or partial or total colonization is successful, and efficacy is observed. In certain embodiments, the composition of the invention can be administered to a pregnant animal, for example a mammal such as a human in order to prevent a condition from developing in her child in utero and/or after it is born.

The compositions of the invention may be administered to a patient that has been diagnosed with a disease or condition mediated histone deacetylase activity, or that has been identified as being at risk of a disease or condition mediated by histone deacetylase activity. The compositions may also be administered as a prophylactic measure to prevent the development of diseases or conditions mediated by histone deacetylase activity in a healthy patient

The microbial-containing and/or EV-containing compositions disclosed herein can be administered to a subject that has been identified as having an abnormal gut microbiota. For example, the patient may have reduced or absent colonization by Barnesiella intestinihominis, Alistipes onderdonkii, Bacteroides finegoldii, Bacteroides vulgatus, and/or an Oscillibacter sp.

The microbial-containing and/or EV-containing compositions disclosed herein can be administered as a food product, such as a nutritional supplement

Generally, the microbial-containing and/or EV-containing compositions disclosed herein are for the treatment of human subjects, although they may be used to treat animals including monogastric mammals such as poultry, pigs, cats, dogs, horses or rabbits or multigastric animals such as ruminants. The compositions of the invention may be useful for enhancing the growth and performance of animals. If administered to animals, oral gavage may be used.

### IV. Kits

Further provided herein are kits containing one or more of microbial strains and/or EVs derived from one or more of the microbial strains disclosed herein. The kits can include one or more of (such as any of 1, 2, 3, or 4, ) strains and/or EVs derived from one or more of the microbial strains provided herein including an Oscillibacter sp. (for example Oscillibacter sp. strain DSM 34011), a B. intestinihominis strain (for example B. intestinihominis strain DSM 34032 and/or B. intestinihominis strain DSM 34012), an A. onderdonkii strain (for example A. onderdonkii strain DSM 34033), a B. finegoldii strain (for example B. finegoldii strain DSM 34013), a B. vulgatus strain (for example B. vulgatus strain DSM 34030), and/or a Oscillibacter sp. strain (for example Oscillibacter sp. strain DSM 34011) along with instructions for proper storage, maintenance, and use for administering to a subject for the treatment or prevention of one or more obesity related disorders. In additional embodiments, the kit further includes one or more strains (such as any of about 1, 2, 3, 4, 5, 6, 7, or 8 or more strains) of E. eligens, I. massiliensis, P. copri, and/or Akkermansia sp. (such as those disclosed in International Patent Application Publication No. WO2021203083, incorporated by reference herein); and/or one or more strains (such as any of about 1, 2, 3, 4, 5, 6, 7, or 8 or more strains) disclosed herein in Table 1 of Example 1.

The invention can be further understood by reference to the following examples, which are provided by way of illustration and are not meant to be limiting.

### EXAMPLES

### Example 1: Discovery of Probiotics for Metabolic Health

This Example details a study to identify probiotics for improvement of metabolic health.

### Materials and Methods

A clinical study was conducted to assess differences in the intestinal microbiota between lean and obese subjects to identify next-generation probiotic (NGP) candidates correlated with improved metabolic health (ClinicalTrials.gov identifier: NCT04229082). The lean subjects recruited to the study had a body mass index (BMI) between 18-25, waist circumference <80 cm and fasting plasma glucose <6.1 mmol/l (n=55). The obese subjects had a BMI between 27.5-39.9, waist circumference >80 cm and/or fasting plasma glucose between 6.1 mmol/1-7.0 mmol/l (n=38). Subjects with BMI between 25-27.5 were additionally accepted into the obese group under the condition that their waist circumference was >85 cm. Body composition of the subjects was measured by BMI and percent whole body fat by dual-energy x-ray absorptiometry (DXA) (Lunar Prodigy Advance, GE Lunar, Madison, WI, USA, Software version enCORE 16) during the study. Metabolic markers of pre-diabetes and obesity, including insulin and glucose levels, were analyzed from blood samples using routing clinical chemistry and hematology. Fecal samples were obtained from the subjects on study visit day 1 (V1) and subsequently at study day 7 (V3) for analysis of the microbiota populations between study groups.

For analytical comparisons, the subjects were further assigned to subgroups within the overall lean and healthy study groups according to their laboratory values for relevant markers of metabolic heath. The definition of a high laboratory value for subgroup determination was a value equal to or greater than the upper quartile (75th percentile) of the observations within the visit.

The study subgroups were defined as follows:
Prediabetic group: Subjects that have high glucose and insulin values on all laboratory measurements (screening and end of study visit).
High glucose group: Subjects that have high glucose values on all laboratory measurements (screening and end of study visit).
High insulin group: Subjects that have high insulin values on all laboratory measurements (screening and end of study visit).
High cholesterol group: cholesterol equal to or greater than 5.0 on all measurements (screening and end of study visit).
High blood pressure group: Systolic blood pressure equal to or greater than 130 or diastolic blood pressure equal to or greater than 85 at screening.
Healthy group: Subjects that do not have high glucose or insulin at any measurement, have cholesterol less than 5.0 on all measurements, have systolic blood pressure less than 130 and diastolic blood pressure less than 85 at screening.

All work involving human subjects was approved by the DuPont Human Study Committee. The study was conducted in compliance with Good Clinical Practices and in accordance with the Declaration of Helsinki.

Sequencing of fecal microbiota. Fecal samples obtained from the clinical study subjects were stored at −80°° C. until processing. Microbial DNA was isolated from 200 mg of the fecal samples using the Qiagen PowerSoil Isolation kit on the KingFisher DNA extraction robot (Thermo Fisher Scientific). Microbiota taxonomic profiles were generated using 16S rRNA amplicon sequencing (Caporaso et al., 2012). The V4 variable region of the 16S rRNA gene was amplified by barcoded PCR primers 515F (5′-GTGCCAGCMGCCGCGGTAA) and 806R (5′-GGACTACHVGGGTWTCTAAT) under the following conditions: denaturation at 95° C. for 3 min followed by 30 cycles of denaturation at 95° C. for 45 s, annealing at 55° C. for 60 s and extension at 72° C. for 90 s, and a final extension at 72° C. for 10 min. PCR products were sequenced using the MiSeq V2 platform with 2×250 nt reads (Illumina). Sequencing data were analyzed using the QIIME pipeline (Caporaso et al., 2010) using a minimum of 10,000 high quality (Phred score >q20) reads per sample. Sequences were clustered into operational taxonomic units (OTUs) at 99% similarity and assigned taxonomy based on the RDP Classifier v 2.13 (Wang et al., 2007). Shotgun metagenomic sequencing was additionally conducted using Illumina HiSeq 2000 (Illumina) 2×150 nt reads with the same DNA samples, and the microbiota composition of the samples was determined by targeted assembly of conserved marker genes (16S rRNA and rplB) and MetaPhlAn (v.1) software (Segata et al., 2012; Wang et al., 2015).

### Results

NGP candidates were identified through differential abundance testing of the microbiota taxa derived from 16S rRNA amplicon and metagenomic sequencing analyses between lean and obese study groups and lean-healthy/obese-prediabetic subgroups. Nonparametric Wilcoxon rank sum tests were conducted between groups for each timepoint individually to identify the differential taxa between the study groups. A broad list of potential probiotic candidates was compiled and included operational OTUs from 16S rRNA amplicon sequencing as well as from shotgun metagenomics analysis where bacterial species were identified based on 16S rRNA and rplB marker genes and MetaPhlAn software (Segata et al., 2012). In total, the NGP candidate list was comprised of 79 OTUs or species that were increased in abundance in the lean-healthy compared to obese prediabetic subgroups (LH vs. OP; p<0.1) or overall lean vs. obese groups (L vs. H; p<0.1) at both visits (Table 1).

TABLE 1
Probiotic candidates with increased abundance in lean and metabolically healthy individuals

| | | | | | | Similarity |
|---|---|---|---|---|---|---|
| Rank | Analysis type | Comparison^{a} | Presumptive taxonomy | OTU ID | Closest reference sequence^{b} | (%) |
| 1 | 16S V4 | L vs. O; LH vs. | Oscillibacter sp. | 1062061 | Oscillibacter valericigenes str. | 94.4 |
| | amplicon | OP | | | Sjm18-20 Type | |
| 2 | 16S V4 | L vs. O; LH vs. | Firmicutes sp. | 584433 | Sedimentibacter hongkongensis str. | 90.2 |
| | amplicon | OP | | | HKU2 Type | |
| 3 | 16S V4 | L vs. O; LH vs. | Clostridiales sp. | 841691 | Ruminiclostridium cellobioparum str. | 92.9 |
| | amplicon | OP | | | DSM: 5398 Type | |
| 4 | 16S V4 | L vs. O; LH vs. | Clostridia sp. | 908728 | Abyssivirga alkaniphila str. L81 Type; | 90.8 |
| | amplicon | OP | | | Vallitalea guaymasensis str. | |
| | | | | | Ra1766G1 Type | |
| 5 | 16S V4 | L vs. O; LH vs. | Clostridia sp. | 1110312 | Criibacterium bergeronii str. | 90.2 |
| | amplicon | OP | | | CCRI: 22567 Type | |
| 6 | 16S V4 | L vs. O | Barnesiella | 840769 | Barnesiella intestinihominis str. YIT | 98.4 |
| | amplicon | | intestinihominis | | 11860 | |
| 7 | 16S V4 | L vs. O; LH vs. | Oscillospiraceae sp. | 559063 | Tepidibaculum saccharolyticum str. | 92.8 |
| | amplicon | OP | | | STR9 Type | |
| 8 | 16S V4 | L vs. O; LH vs. | Intestinimonas | 843022 | Intestinimonas massiliensis str. GD2 | 100 |
| | amplicon | OP | massiliensis | | Type | |
| 9 | 16S V4 | L vs. O; LH vs. | Oscillospiraceae sp. | 739971 | Dysosmobacter welbionis str. J115 | 95.7 |
| | amplicon | OP | | | Type | |
| 10 | 16S V4 | L vs. O; LH vs. | Firmicutes sp. | 548720 | Bacillus thermocloacae str. DSM | 88.8 |
| | amplicon | OP | | | 5250 Type | |
| 11 | 16S V4 | L vs. O; LH vs. | Clostridia sp. | OTU224 | Intestinimonas massiliensis str. GD2 | 91.3 |
| | amplicon | OP | | | Type | |
| 12 | 16S V4 | LH vs. OP | Prevotella copri | 909561 | Prevotella copri str. JCM: 13464 Type | 100 |
| | amplicon | | | | | |
| 13 | 16S V4 | L vs. O | Oscillibacter sp. | 1099678 | Oscillibacter valericigenes str. | 94.8 |
| | amplicon | | | | Sjm18-20 | |
| 14 | 16S V4 | L vs. O | Acutalibacter sp. | 370112 | Acutalibacter muris str. KB18 Type | 96 |
| | amplicon | | | | | |
| 15 | 16S V4 | L vs. O | Sporobacter sp. | 843886 | Sporobacter termitidis str. SYR Type | 94 |
| | amplicon | | | | | |
| 16 | 16S V4 | L vs. O | Clostridia sp. | 331966 | Clostridium chartatabidum str. DSM | 91.3 |
| | amplicon | | | | 5482 Type | |
| 17 | 16S V4 | L vs. O | Sporobacter sp. | 359720 | Sporobacter termitidis str. SYR Type | 94.9 |
| | amplicon | | | | | |
| 18 | 16S V4 | L vs. O | Clostridia sp. | 573053 | Desulfosporosinus burensis str. | 89.4 |
| | amplicon | | | | BSREI1 Type | |
| 19 | 16S V4 | LH vs. OP | Lachnospira eligens | 850488 | Lachnospira eligens_L34420 | 99.6 |
| | amplicon | | | | | |
| 20 | 16S V4 | L vs. O | Oscillospiraceae sp. | 806064 | Dysosmobacter welbionis str. J115 | 94.5 |
| | amplicon | | | | Type | |
| 21 | Metagenome | LH vs. OP | Acholeplasma | Acholeplasma_52 | | |
| | rplB | | | | | |
| 22 | Metagenome | LH vs. OP | Candidatus Soleaferrea | Candidatus | | |
| | rplB | | | Soleaferrea_314 | | |
| 23 | Metagenome | LH vs. OP | Clostridium | Clostridium_9 | | |
| | rplB | | | | | |
| 24 | Metagenome | LH vs. OP | Eubacterium eligens | Eubacterium_eligens | | |
| | MetaPhlAn | | | | | |
| 25 | 16S V4 | L vs. O | Oscillospiraceae sp. | 586517 | Thermoclostridium caenicola str. | 90 |
| | amplicon | | | | EBR596 Type | |
| 26 | 16S V4 | L vs. O | Alistipes obesi | 575041 | Alistipes obesi str. ph8 Type | 100 |
| | amplicon | | | | | |
| 27 | 16S V4 | LH vs. OP | Agathobaculum | 580521 | Agathobaculum desmolans str. | 99.6 |
| | amplicon | | desmolans | | ATCC: 43058 Type | |
| 28 | 16S V4 | LH vs. OP | Oscillospiraceae sp. | 734826 | Lawsonibacter asaccharolyticus str. | 94 |
| | amplicon | | | | 3BBH22 Type | |
| 29 | 16S V4 | LH vs. OP | Alistipes inops | 4336943 | Alistipes inops str. 627 Type | 100 |
| | amplicon | | | | | |
| 30 | 16S V4 | L vs. O | Akkermansia sp. | 192963 | Akkermansia muciniphila str. | 97.6 |
| | amplicon | | | | ATCC: BAA-835 Type | |
| 31 | 16S V4 | L vs. O | Colidextribacter sp. | 365161 | Colidextribacter massiliensis str. | 97.6 |
| | amplicon | | | | Marseille-P3083 Type | |
| 32 | 16S V4 | L vs. O | Clostridia sp. | 831922 | Desulfosporosinus burensis str. | 89 |
| | amplicon | | | | BSREI1 | |
| 33 | 16S V4 | LH vs. OP | Adlercreutzia sp. | 303498 | Adlercreutzia muris str. WCA-131- | 94.8 |
| | amplicon | | | | CoC-2 Type | |
| 34 | 16S V4 | LH vs. OP | Clostridia sp. | 549577 | Lutispora thermophila str. EBR46 | 92 |
| | amplicon | | | | Type | |
| 35 | 16S V4 | LH vs. OP | Prevotella copri | 588173 | Prevotella copri str. JCM: 13464 Type | 99.6 |
| | amplicon | | | | | |
| 36 | Metagenome | LH vs. OP | Alistipes | Alistipes_457 | | |
| | rplB | | | | | |
| 37 | Metagenome | LH vs. OP | Anaerotruncus | Anaerotruncus_45 | | |
| | rplB | | | | | |
| 38 | Metagenome | LH vs. OP | Eubacterium | Eubacterium_194 | | |
| | rplB | | | | | |
| 39 | Metagenome | LH vs. OP | Eubacterium siraeum | Eubacterium_siraeum | | |
| | MetaPhlAn | | | | | |
| 40 | Metagenome | LH vs. OP | Prevotella copri | Prevotella_copri | | |
| | MetaPhlAn | | | | | |
| 41 | 16S V4 | L vs. O | Clostridia sp. | 538796 | Hungatella effluvii str. UB-B.2 Type | 90.2 |
| | amplicon | | | | | |
| 42 | 16S V4 | L vs. O | Oscillospiraceae sp. | 558420 | Ruminiclostridium cellobioparum str. | 90.9 |
| | amplicon | | | | DSM 1351 Type | |
| 43 | 16S V4 | LH vs. OP | Clostridia sp. | 362450 | Christensenella massiliensis str. | 88.8 |
| | amplicon | | | | Marseille-P2438 Type | |
| 44 | 16S V4 | LH vs. OP | Prevotella copri | 849625 | Prevotella copri str. JCM: 13464 Type | 99.6 |
| | amplicon | | | | | |
| 45 | 16S V4 | LH vs. OP | Prevotella copri | 1045110 | Prevotella copri str. JCM: 13464 Type | 99.6 |
| | amplicon | | | | | |
| 46 | Metagenome | LH vs. OP | Acetivibrio | Acetivibrio_1297 | | |
| | 16S | | | | | |
| 47 | Metagenome | LH vs. OP | Aeribacillus | Aeribacillus_77 | | |
| | rplB | | | | | |
| 48 | Metagenome | LH vs. OP | Bacteroidales | Bacteroidales_{—} | | |
| | MetaPhlAn | | bacterium_ph8 | bacterium_ph8 | | |
| 49 | Metagenome | LH vs. OP | Butyrivibrio | Butyrivibrio_260 | | |
| | rplB | | | | | |
| 50 | Metagenome | LH vs. OP | Holdemanella | Holdemanella_200 | | |
| | rplB | | | | | |
| 51 | Metagenome | LH vs. OP | Holdemanella | Holdemanella_39 | | |
| | 16S | | | | | |
| 52 | Metagenome | LH vs. OP | Oscillibacter | Oscillibacter_14 | | |
| | rplB | | | | | |
| 53 | 16S V4 | L vs. O | Oscillospiraceae sp. | 302165 | Sporobacter termitidis str. SYR Type | 93.6 |
| | amplicon | | | | | |
| 54 | Metagenome | LH vs. OP | Catabacter | Catabacter_207 | | |
| | rplB | | | | | |
| 55 | Metagenome | LH vs. OP | Clostridium | Clostridium_472 | | |
| | rplB | | | | | |
| 56 | Metagenome | LH vs. OP | Clostridium | Clostridium_834 | | |
| | 16S | | | | | |
| 57 | 16S V4 | L vs. O | Clostridia sp. | 295615 | Intestinimonas butyriciproducens | 92.3 |
| | amplicon | | | | str. DSM: 26588 Type | |
| 58 | 16S V4 | L vs. O | Mollicutes sp. | 360268 | Acholeplasma parvum str. H23M | 90.7 |
| | amplicon | | | | Type | |
| 59 | 16S V4 | LH vs. OP | Prevotella copri | 511813 | Prevotella copri str. JCM: 13464 Type | 99.6 |
| | amplicon | | | | | |
| 60 | 16S V4 | LH vs. OP | Prevotella copri | 529745 | Prevotella copri str. JCM: 13464 Type | 99.6 |
| | amplicon | | | | | |
| 61 | Metagenome | LH vs. OP | Clostridium | Clostridium_302 | | |
| | 16S | | | | | |
| 62 | Metagenome | LH vs. OP | Clostridium | Clostridium_755 | | |
| | 16S | | | | | |
| 63 | Metagenome | LH vs. OP | Dialister | Dialister_183 | | |
| | rplB | | | | | |
| 64 | Metagenome | LH vs. OP | Domibacillus | Domibacillus_4 | | |
| | rplB | | | | | |
| 65 | Metagenome | LH vs. OP | Ethanoligenens | Ethanoligenens_284 | | |
| | 16S | | | | | |
| 66 | Metagenome | LH vs. OP | Unclassified | Lachnospiraceae_{—} | | |
| | MetaPhlAn | | Lachnospiraceae | bacterium_3_1_46FAA | | |
| 67 | Metagenome | LH vs. OP | Oribacterium | Oribacterium_263 | | |
| | rplB | | | | | |
| 68 | Metagenome | LH vs. OP | Parabacteroides | Parabacteroides_merdae | | |
| | MetaPhlAn | | merdae | | | |
| 69 | Metagenome | LH vs. OP | Ruminiclostridium | Ruminiclostridium_326 | | |
| | 16S | | | | | |
| 70 | 16S V4 | L vs. O | Hungatella sp. | OTU147 | Hungatella xylanolytica str. | 96.8 |
| | amplicon | | | | CCUG: 48289T Type | |
| 71 | 16S V4 | L vs. O | Eubacterium sp. | 365891 | Eubacterium oxidoreducens str. | 97.2 |
| | amplicon | | | | DSM: 3217 Type | |
| 72 | Metagenome | LH vs. OP | Streptococcus australis | Streptococcus_australis | | |
| | MetaPhlAn | | | | | |
| 73 | Metagenome | LH vs. OP | Lutispora | Lutispora_352 | | |
| | 16S | | | | | |
| 74 | Metagenome | LH vs. OP | Paraprevotella | Paraprevotella_917 | | |
| | 16S | | | | | |
| 75 | Metagenome | LH vs. OP | Acholeplasma | Acholeplasma_76 | | |
| | rplB | | | | | |
| 76 | Metagenome | LH vs. OP | Butyricicoccus | Butyricicoccus_1305 | | |
| | 16S | | | | | |
| 77 | Metagenome | LH vs. OP | Butyricimonas | Butyricimonas_429 | | |
| | rplB | | | | | |
| 78 | Metagenome | LH vs. OP | Gemmiger | Gemmiger_1058 | | |
| | 16S | | | | | |
| 79 | Metagenome | LH vs. OP | Eubacterium | Eubacterium_51 | | |
| | rplB | | | | | |

^{a}L vs. O, Lean vs. Obese group comparison; LH vs. OP, Lean-healthy vs. Obese prediabetic subgroup comparison.

### Example 2: Selection of Priority Candidate Strains for Probiotic Use

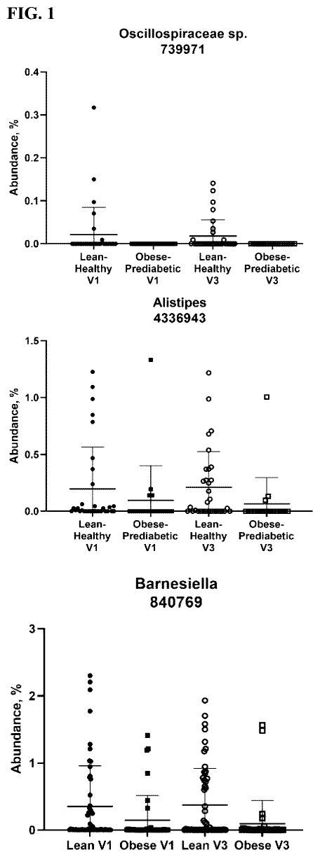

The NGP candidates were prioritized by ranking the candidates according to the following criteria: abundance of the taxon in the lean-healthy or lean study groups (FIG. 1) and correlation analyses with subject clinical biomarkers (Insulin, BMI, Glucose, DXA total fat mass) by Spearman correlation coefficient analysis (Table 2).

TABLE 2
Abundance of priority next generation probiotic (NGP) candidates in healthy subjects and their correlation to clinical metabolic health markers

| | | | | Abundance | | | | | | | | |
|---|---|---|---|---|---|---|---|---|---|---|---|---|
| | | | | in lean- | | | | | | | | |
| | | | Average | healthy vs. | | | | | | | | |
| | | | abundance | obese- | Spearman correlation coefficient (rho)^{c} | | | | | | | |
| | | | in lean- | prediabetic | | | | | | | V1 DXA | V3 DXA |
| | | | healthy | (log2 fold | V1 | V3 | V1 | V3 | V1 | V3 | Total Fat | Total Fat |
| Rank | OTU | Taxonomy | group (%) | change) | Insulin | Insulin | BMI | BMI | Glucose | Glucose | Mass | Mass |
| 6 | 840769 | Barnesiella | 0.38^{a} | 1.98^{b} | −0.22* | −0.26* | −0.22* | −0.3** | −0.1 | −0.25* | −0.27** | −0.33*** |
| | | intestinihominis | | | | | | | | | | |
| 9 | 739971 | Oscillospiraceae | 0.01 | 5.81 | −0.25* | −0.18⁺ | −0.15 | −0.15 | −0.17⁺ | −0.2* | −0.17 | −0.19⁺ |
| | | sp. | | | | | | | | | | |
| 29 | 4336943 | Alistipes inops | 0.21 | 1.7 | −0.09 | −0.27** | −0.04 | −0.07 | −0.12 | −0.16 | −0.05 | −0.12 |

^{a}Taxon abundance in overall lean group.
^{b}log₂ fold change in overall lean group compared to obese group.
^{c}Correlation between taxon abundance and metabolic marker values (Spearman correlation coefficient);
*p < 0.05,
**p < 0.01,
***p < 0.001,
⁺<0.1.

### Example 3: Metabolic Capabilities of Priority Candidate Strains

This Example examined the production of a number of metabolites known to impact metabolic health and to promote viral resistance by the priority candidate strains identified in Example 2.

### Materials and Methods

Fecal samples from healthy subjects (ClinicalTrials.gov identifier: NCT04229082) were used to isolate bacterial species and a selected group of isolates were examined for their metabolic capabilities. For comparison of metabolic capabilities of the Barnesiella intestinihominis DSM34032, Barnesiella intestinihominis DSM34012, Alistipes onderdonkii DSM34033, Bacteroides finegoldii DSM34013, Phocaeicola vulgatus/Bacteroides vulgatus DSM34030 and Oscillibacter sp. DSM34011 strains were grown in YCFAC media. YCFAC media was inoculated with 1% overnight culture and supernatants were collected after 24 hrs of growth. Cells were separated by centrifugation at 10,000 rpm for 5 mins and then filtered by 0.2 μM filter.

At end of 24 hrs of growth cell-free supernatants was harvested and analyzed by CE-TOF-MS. Cationic conditions: Samples was injected using 50 mbar, 10 sec onto Fused Silica Capillary (i.d. 50 μm×0 cm) on an Agilent CE-TOF System (Agilent Technologies Inc., Santa Clara, CA, USA). Cationic Buffer solution (1M Formic acid) was used and a CE-voltage of 30 kV. Positive mode mass spectrometer conditions: MS Capillary voltage: 4.0V, ESI Positive ionization mode, m/z range 50-1000. Anionic conditions: Samples was injected using 50 mbar, 22 sec onto Fused Silica Capillary (i.d. 50 μm×0 cm) on an Agilent CE-TOF System (Agilent Technologies Inc.). Anionic Buffer solution (50 mM Ammonium acetate at pH=7.5) was used and a CE-voltage of 30kV. Negative mode mass spectrometer conditions: MS Capillary voltage: 3.5V, ESI Negative ionization mode, m/z range 50-1000.

The metabolites agmatine (N-(4-aminobutyl)guanidine) and carnosine were detected in cationic mode at retention time RT-4.23 mins at m/z 131.130 m.u. and RT=5.56 mins at m/z 227.115 m.u. respectively. Penciclovir (RT=8.38 mins, m/z 254.125 m.u., cationic mode), Octanoic acid (RT=7.33 mins, m/z 143.109 m.u., anionic mode), tripeptide Ile-Pro-Pro (RT=8.81 mins, m/z 326.209 m.u., cationic mode) and Val-Pro-Pro (RT-8.67 mins, m/z 312.193 m.u., cationic mode) were detected using the CE-TOF system.

The metabolites 2-oxo-isovaleric acid (3-methyl-2-oxo-butanoic acid) and 3-methyl-2-oxo-valeric acid (3-methyl-2-oxo-pentanoic acid) were detected in the anionic mode at retention time 8.84 mins at m/z 115.041 m.u.

Glutathione (reduced form, GSH, gamma-L-glutamyl-L-cysteinyl-glycine) was determined in cationic mode at RT=10.64 mins at m/z 308.093 m.u. and GABA (4-amino-butyric acid) in cationic mode at RT=6.24 mins at m/z 104.071 m.u.

The identification was performed against a known standard. The reported amounts are peak arcas.

At end of 24 hrs of growth cell-free supernatants was harvested and analyzed by Nuclear Magnetic Resonance (NMR) Spectroscopy. 100 μL sample are mixed with 100L 750 mM Phosphate-buffer in D₂O solution with 1.0029 mM DSS-d6 as an internal standard and transferred to a 3 mm NMR tube. 1H NMR measurements were performed at 298K on a 600 MHz Avance III spectrometer (Bruker Biospins, Rheinstetten, Germany) and equipped with a 5 mm TCI CryoProbe. A standard ID NOESY experiment with pre-saturation (Bruker ‘noesyprld’ sequence) was used to acquire 1H NMR spectra. A total of 128 scans collected into 64K data points were obtained with a spectral width of 20.03 ppm, a recycle delay of 3 s and an acquisition time of 2.73 s. All 1H spectra were processed with an exponential line-broadening of 0.3 Hz prior to the Fourier transformation. Each spectrum was automatically phased and referenced to the DSS signal at 0 ppm using TopSpin 3.2 (Bruker BioSpin, Rheinstetten, Germany). The Chenomx NMR Suite 8.5 (Chenomx, Edmonton, Canada) software was applied to assign and quantify metabolites by determining the area of each metabolite and comparing the area with the integral of the DSS signal. In addition, a 1H-13C heteronuclear single quantum coherence (HSQC; hsqcedetgpsisp2.3, Bruker pulse sequence) experiment was performed on a pooled sample for metabolite assignment. The metabolites formic, acetic, propionic and succinic acid were quantified using NMR.

### Results

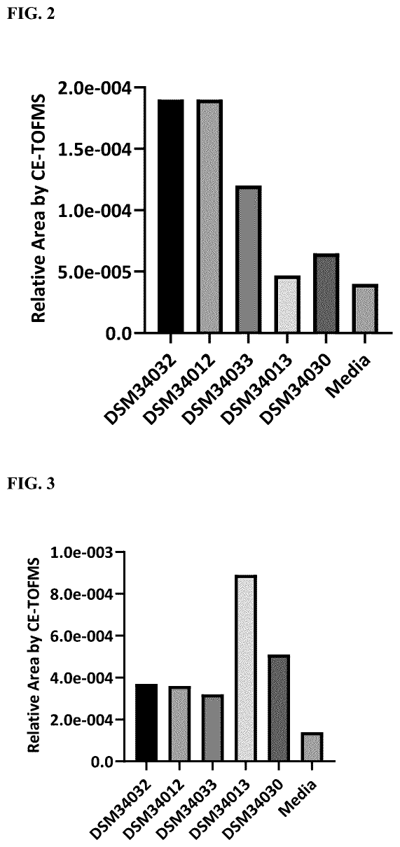

Agmatine is metabolized from arginine by the enzyme arginine decarboxylase EC 4.1.1.19 (Piletz et al 2013, Taksande et al 2016). From the data shown in FIG. 2, the level of agmatine is higher in Barnesiella intestinihominis DSM34032, Barnesiella intestinihominis DSM34012, Alistipes onderdonkii DSM34033, Bacteroides finegoldii DSM34013, and Bacteroides vulgatus DSM34030 as compared to the YCFAC growth media.

Carnosine (beta-alanyl-L-histidine) is a dipeptide molecule, made up of the amino acids beta-alanine and histidine. From the data shown in FIG. 3, the level of carnosine is higher in Barnesiella intestinihominis DSM34032, Barnesiella intestinihominis DSM34012, Alistipes onderdonkii DSM34033, Bacteroides finegoldii DSM34013, and Bacteroides vulgatus DSM34030 as compared to the YCFAC growth media.

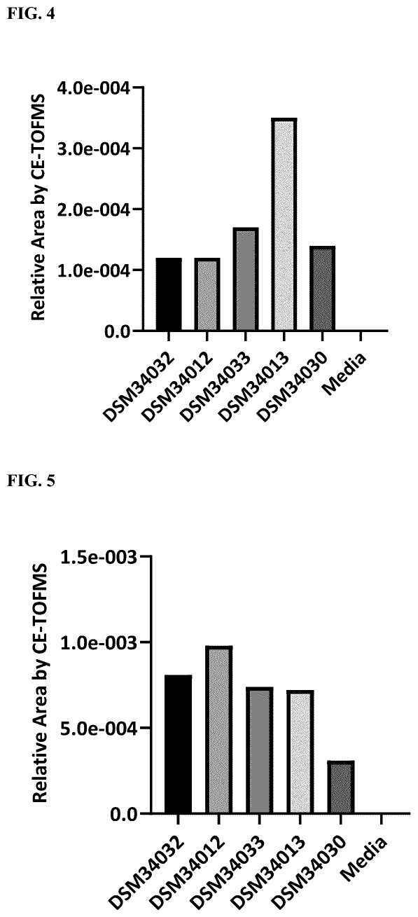

Penciclovir (Synonyms: CAS-nr: 39809-25-1, Denavir, Penciclovirum) is a guanosine analogue and has been shown to have antiviral properties. From the data shown in FIG. 4, the level of penciclovir is higher in Barnesiella intestinihominis DSM34032, Barnesiella intestinihominis DSM34012, Alistipes onderdonkii DSM34033, Bacteroides finegoldii DSM34013, and Bacteroides vulgatus DSM34030 as compared to the YCFAC growth media. The measured difference between the strains and the media level indicates that Barnesiella intestinihominis DSM34032, Barnesiella intestinihominis DSM34012, Alistipes onderdonkii DSM34033, Bacteroides finegoldii DSM34013, and Bacteroides vulgatus DSM34030 are producing penciclovir.

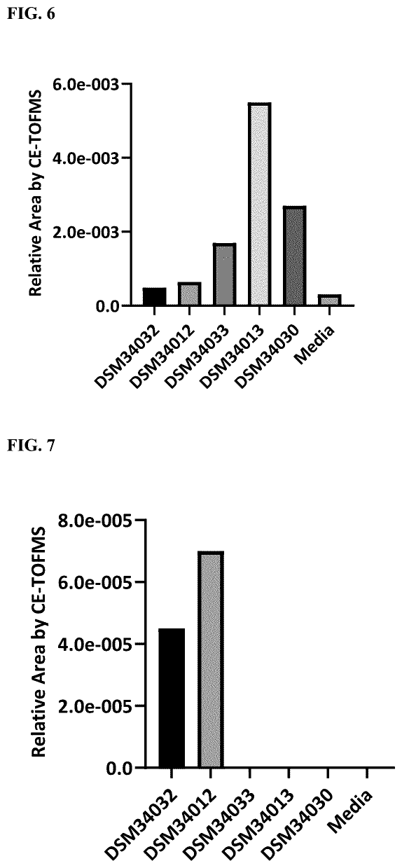

Ile-Pro-Pro and Val-Pro-Pro are bioactive tripeptides that can be used to prevent metabolic disorders and to assist in cardiovascular health management. From the data shown in FIG. 5 and FIG. 6, the level of tripeptides is higher in Barnesiella intestinihominis DSM34032, Barnesiella intestinihominis DSM34012, Alistipes onderdonkii DSM34033, Bacteroides finegoldii DSM34013, and Bacteroides vulgatus DSM34030 as compared to the YCFAC growth media. The measured difference between the strains and the media level indicates that Barnesiella intestinihominis DSM34032, Barnesiella intestinihominis DSM34012, Alistipes onderdonkii DSM34033, Bacteroides finegoldii DSM34013, and Bacteroides vulgatus DSM34030 are producing the Ile-Pro-Pro and Val-Pro-Pro tripeptides.

Octanoic acid (a.k.a. caprylic acid), is a medium-chain fatty acid with potent antibacterial, antifungal, and anti-inflammatory properties. These properties make octanoic acid a helpful treatment for many conditions such as high cholesterol. From the data shown in FIG. 7, the level of octanoic acid is higher in Barnesiella intestinihominis DSM34032 and Barnesiella intestinihominis DSM34012 as compared to the YCFAC growth media. The measured difference between the strains and the media level indicates that Barnesiella intestinihominis DSM34032 and Barnesiella intestinihominis DSM34012 are producing octanoic acid.

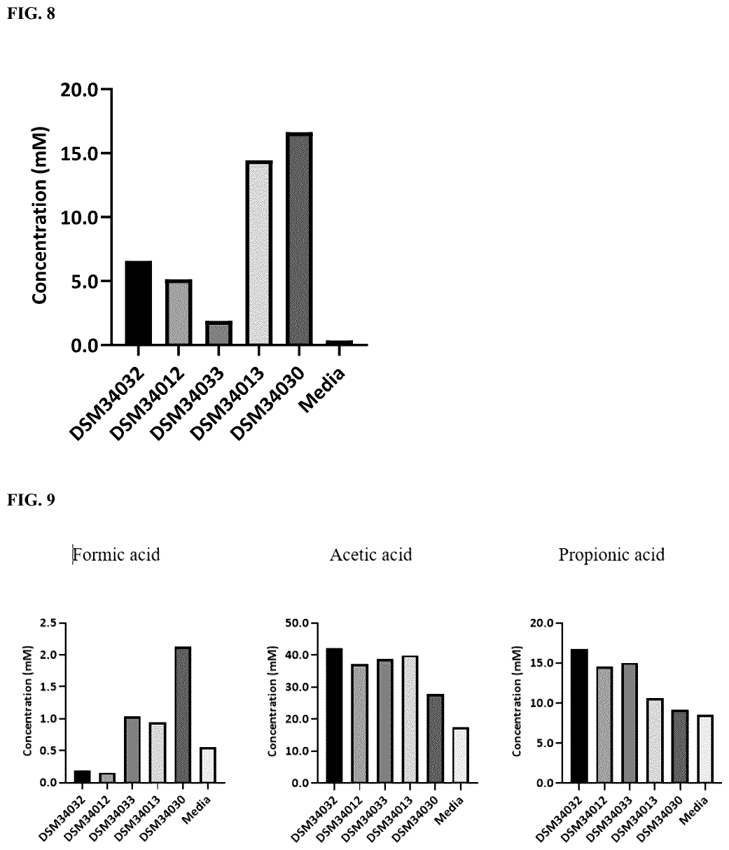

Succinic acid is produced as part of central carbon metabolism in cells. The molecule can serve as a promotor of growth of probiotics in the colon mitigating gut dysbiosis and interacts with the host/human through the SUCNRI receptor which has been associated with anti-inflammatory properties and signaling associated with metabolic diseases (Fernandez-Veledo and Vendrell, 2019). These properties make succinic acid a helpful treatment for many conditions such as hypertension, diabetes, liver fibrosis, non-alcoholic fatty liver disease. From the data shown in FIG. 8, the level of succinic acid is higher in Barnesiella intestinihominis DSM34032, Barnesiella intestinihominis DSM34012, Alistipes onderdonkii DSM34033, Bacteroides finegoldii DSM34013, and Bacteroides vulgatus DSM34030 as compared to the YCFAC growth media.

Short chain fatty acids play a central role in health. From the data shown in FIG. 9, the level of formic acid is higher Alistipes onderdonkii DSM34033, Bacteroides finegoldii DSM34013, and Phocaeicola vulgatus/Bacteroides vulgatus DSM34030 as compared to the YCFAC growth media. The level of acetic and propionic acid is higher in Barnesiella intestinihominis DSM34032, Barnesiella intestinihominis DSM34012, Alistipes onderdonkii DSM34033, Bacteroides finegoldii DSM34013, and Phocaeicola vulgatus/Bacteroides vulgatus DSM34030 as compared to the YCFAC growth media.

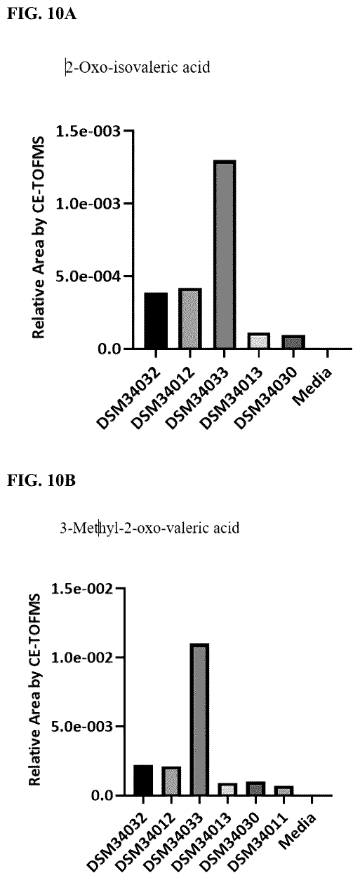

Branched-chain keto acids (BCKAs) are degradation product from branched-chain amino acids. The degradation of BCKAs is related to insulin resistance and glucose intolerance. 2-oxo-isovaleric acid is the degradation product of valine while 3-methyl-2-oxo-valeric acid is the degradation product of isoleucine. Both reactions are catalyzed by the enzyme branched-chain amino acid aminotransfercase (BCAT). From the data shown in FIG. 10A and/or FIG. 10B, the level of BCKAs is higher in Barnesiella intestinihominis DSM34032, Barnesiella intestinihominis DSM34012, Bacteroides finegoldii DSM34013, Bacteroides vulgatus DSM34030, Oscillibacter sp. DSM34011, and, in particular, Alistipes onderdonkii DSM34033 as compared to the YCFAC growth media. The measured difference between the strains and the media level indicates that Barnesiella intestinihominis DSM34032, Barnesiella intestinihominis DSM34012, Alistipes onderdonkii DSM34033, Bacteroides finegoldii DSM34013, Bacteroides vulgatus DSM34030, and Oscillibacter sp. DSM34011 are producing BCKAs by degrading branched chain amino acids.

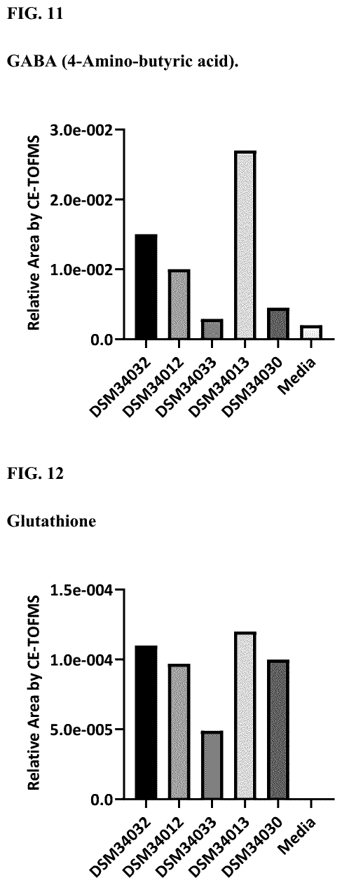

GABA (4-amino-butyric acid) is produced from the amino acid glutamic acid which is catalyzed by the enzyme glutamate decarboxylase. This molecule is a neurotransmitter and an endocrine mediator which influences gut motility. From the data shown in FIG. 11, the level of GABA s is higher in Barnesiella intestinihominis DSM34032, Barnesiella intestinihominis DSM34012, Alistipes onderdonkii DSM34033, Bacteroides finegoldii DSM34013, and Bacteroides vulgatus DSM34030 as compared to the YCFAC growth media.

Glutathione is a thiol group-containing tripeptide making it a natural antioxidant making it useful for the treatment of liver diseases like non-alcoholic fatty liver disease (NAFLD) and liver steatosis (NASH; Antonella Borrellia and Franco Maria Buonaguroa, 2018). From the data shown in FIG. 12, the level of Glutathione is higher in Barnesiella intestinihominis DSM34032, Barnesiella intestinihominis DSM34012, Alistipes onderdonkii DSM34033, Bacteroides finegoldii DSM34013, and Bacteroides vulgatus DSM34030 as compared to the YCFAC growth media.

### Example 4: Taxonomic Analysis for Oscillibacter sp. Candidate Strains

This Example analyzed the taxonomy of the candidate Oscillibacter sp. DSM34011 srtain.

### Materials and Methods

Genome Sequencing and Assembly: The genome of the Oscillibacter sp. strain identified in Example 1 was sequenced using Illumina NovaSeq 6000 paired-end 2×250 bp reads. The reads were quality filtered, error corrected using BFC and then assembled using SPAdes (Bankevish et al., 2012; Li, 2015). The assembly was further corrected using Pilon and annotated using Prokka (Seemann, 2014; Walker et al., 2014). The completeness of the genome assembly was evaluated with BUSCO based on gene content from near-universal single-copy orthologs (Simao et al., 2015). The resulting draft genome of the Oscillibacter sp. strain was comprised of 121 contigs with a total genome size of 3.39 Mbp, an N50 of 127,403 and an average G+C content of 55.7%.

### Results

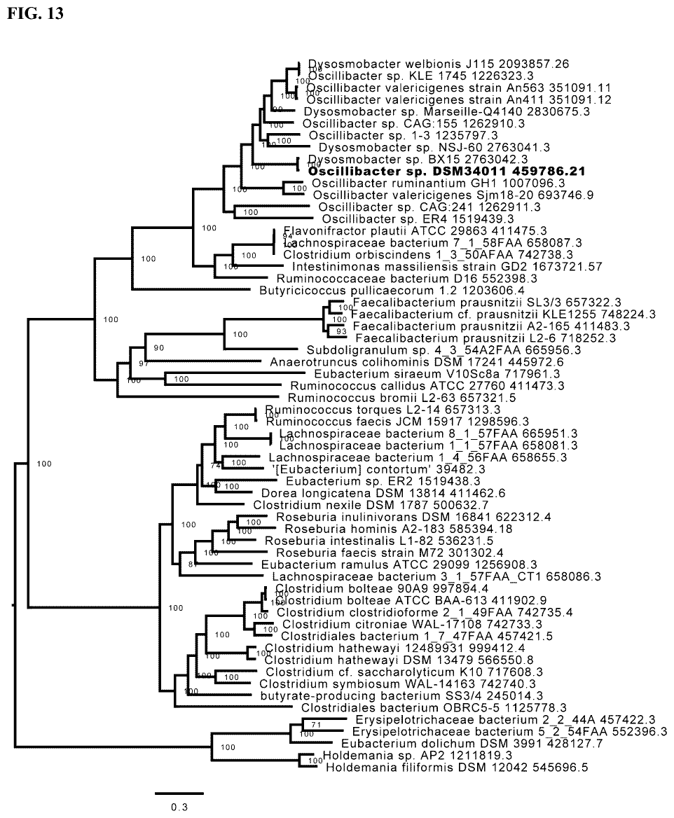

The taxonomy of the Oscillibacter sp. DSM34011 was determined by comparison of the full-length 16S rRNA gene and the genome-wide average nucleotide identity (ANI) to the type strains of closely related species. The 16S rRNA sequence similarity of the closest relatives Dysosmobacter welbionis, Oscillibacter valericigenes and Oscillibacter ruminantium are all greater than the recognized species boundary of 98.6% (Table 3) (Kim et al., 2014). The genome-wide ANI between strain DSM34011and D. welbionis J115 is 81.4%, and 78.0% between strain DSM34011 and O. valericigenes Sjm18-20. The ANI of DSM34011 to its closest relatives is greater than 95%, which is a representative cut-off for speciation (Table 4) (Goris et al., 2007). A phylogenetic tree was constructed containing Oscillibacter-like species including type strains and other publicly available genomes. RAxML was used to align 100 single copy core genes with bootstrap values shown for 100 iterations (FIG. 13) (Stamatakis, 2014). The Oscillibacter sp. DSM34011 strain does not cluster clearly with either the type strains of known species from Oscillibacter or Dysosmobacter. Based on the results from both the taxonomic and phylogenetic analyses, the Oscillibacter sp. DSM34011 is proposed as a novel species.

TABLE 3
16S rRNA similarity for strain DSM34011 to the nearest type strains.

| | Sequence | | |
|---|---|---|---|
| | similarity | | |
| Strain | (%) | NCBI RefSeq ID | Nearest type strain |
| DSM34011 | 0.970 | MG963288 | Dysosmobacter welbionis str. J115 |
| DSM34011 | 0.961 | AB238598 | Oscillibacter valericigenes str. Sjm 18-20 |
| DSM34011 | 0.954 | JF750939 | Oscillibacter ruminantium str. GH1 |
| DSM34011 | 0.933 | NZ_LN869527.1 | Intestinimonas massiliensis str. GD2 |

TABLE 4
Genome-wide average nucleotide identity (ANI) for strain P3_135 to the nearest type strains.

| Strain | DSM34011 | J115 | GH1 | Sjm18-20 |
|---|---|---|---|---|
| Oscillibacter sp. DSM34011 | 100.0 | 81.4 | 78.0 | 77.9 |
| Dysosmobacter welbionis str. J115 | 81.4 | 100.0 | 78.4 | 78.6 |
| Oscillibacter ruminantium str. GH1 | 78.0 | 78.4 | 100.0 | 82.0 |
| Oscillibacter valericigenes str. Sjm18-20 | 77.9 | 78.6 | 82.0 | 100.0 |

### REFERENCES

Caporaso, J. G., Kuczynski, J., Stombaugh, J., Bittinger, K., Bushman, F. D., Costello, E.K., Fierer, N., Pena, A. G., Goodrich, J. K., Gordon, J. I., et al. (2010). QIIME allows analysis of high-throughput community sequencing data. Nat Meth 7, 335-336.
Caporaso, J. G., Lauber, C. L., Walters, W. A., Berg-Lyons, D., Huntley, J., Fierer, N., Owens, S. M., Betley, J., Fraser, L., Bauer, M., et al. (2012). Ultra-high-throughput microbial community analysis on the Illumina HiSeq and MiSeq platforms. ISME J 6, 1621-1624.
Segata, N., Waldron, L., Ballarini, A., Narasimhan, V., Jousson, O., and Huttenhower, C. (2012). Metagenomic microbial community profiling using unique clade-specific marker genes. Nat Methods 9, 811-814.
Wang, Q., Fish, J. A., Gilman, M., Sun, Y., Brown, C. T., Tiedje, J. M., and Cole, J. R. (2015). Xander: employing a novel method for efficient gene-targeted metagenomic assembly. Microbiome 3, 32.
Wang, Q., Garrity, G. M., Tiedje, J. M., and Cole, J. R. (2007). Naive Bayesian classifier for rapid assignment of rRNA sequences into the new bacterial taxonomy. Appl Environ Microbiol 73,5261-5267.
Antonella Borrellia, P. B., Franca Maria Tuccilloa, Ira D. Goldfineb, Joseph L. Evansc,, and Franco Maria Buonaguroa, A. M. (2018). Role of gut microbiota and oxidative stress in the progression of nonalcoholic fatty liver disease to hepatocarcinoma: Current and innovative therapeutic approaches. Redox Biology 15, 467-479.
Caporaso, J. G., Kuczynski, J., Stombaugh, J., Bittinger, K., Bushman, F. D., Costello, E. K., Fierer, N., Pena, A. G., Goodrich, J. K., Gordon, J. I., et al. (2010). QIIME allows analysis of high-throughput community sequencing data. Nat Meth 7, 335-336.
Caporaso, J. G., Lauber, C. L., Walters, W. A., Berg-Lyons, D., Huntley, J., Fierer, N., Owens, S. M., Betley, J., Fraser, L., Bauer, M., et al. (2012). Ultra-high-throughput microbial community analysis on the Illumina HiSeq and MiSeq platforms. ISME J 6, 1621-1624.
Fernandez-Veledo, S., and Vendrell, J. (2019). Gut microbiota-derived succinate: Friend or foe in human metabolic diseases? Rev Endocr Metab Disord 20, 439-447.
Newgard, C. B. (2012). Interplay between lipids and branched-chain amino acids in development of insulin resistance. Cell Metab 15, 606-614.
Segata, N., Waldron, L., Ballarini, A., Narasimhan, V., Jousson, O., and Huttenhower, C. (2012). Metagenomic microbial community profiling using unique clade-specific marker genes. Nat Methods 9, 811-814.
Walejko, J. M., Christopher, B. A., Crown, S. B., Zhang, G. F., Pickar-Oliver, A., Yoneshiro, T., Foster, M. W., Page, S., van Vliet, S., Ilkayeva, O., et al. (2021). Branched-chain alpha-ketoacids are preferentially reaminated and activate protein synthesis in the heart. Nat Commun 12, 1680.
Wang, Q., Fish, J. A., Gilman, M., Sun, Y., Brown, C. T., Tiedje, J. M., and Cole, J. R. (2015). Xander: employing a novel method for efficient gene-targeted metagenomic assembly. Microbiome 3, 32.
Wang, Q., Garrity, G. M., Tiedje, J. M., and Cole, J. R. (2007). Naive Bayesian classifier for rapid assignment of rRNA sequences into the new bacterial taxonomy. Appl Environ Microbiol 73, 5261-5267.
Bankevich, A., Nurk, S., Antipov, D., Gurevich, A. A., Dvorkin, M., Kulikov, A. S., Lesin, V. M., Nikolenko, S. I., Pham, S., Prjibelski, A. D., et al. (2012). SPAdes: a new genome assembly algorithm and its applications to single-cell sequencing. J Comput Biol 19, 455-477.
Goris, J., Konstantinidis, K. T., Klappenbach, J. A., Coenye, T., Vandamme, P., and Tiedje, J. M. (2007). DNA-DNA hybridization values and their relationship to whole-genome sequence similarities. Int J Syst Evol Microbiol 57, 81-91.
Kim, M., Oh, H. S., Park, S. C., and Chun, J. (2014). Towards a taxonomic coherence between average nucleotide identity and 16S rRNA gene sequence similarity for species demarcation of prokaryotes. Int J Syst Evol Microbiol 64, 346-351.
Li, H. (2015). BFC: correcting Illumina sequencing errors. Bioinformatics 31, 2885-2887.
Seemann, T. (2014). Prokka: rapid prokaryotic genome annotation. Bioinformatics 30, 2068-2069.
Simao, F. A., Waterhouse, R. M., Ioannidis, P., Kriventseva, E. V., and Zdobnov, E. M. (2015). BUSCO: assessing genome assembly and annotation completeness with single-copy orthologs. Bioinformatics 31, 3210-3212.
Stamatakis, A. (2014). RAXML version 8: a tool for phylogenetic analysis and post-analysis of large phylogenies. Bioinformatics 30, 1312-1313.
Walker, B. J., Abeel, T., Shea, T., Priest, M., Abouelliel, A., Sakthikumar, S., Cuomo, C. A., Zeng, Q., Wortman, J., Young, S. K., et al. (2014). Pilon: an integrated tool for comprehensive microbial variant detection and genome assembly improvement. PLOS One 9, e112963.

SEQUENCES

Barnesiella intestinihominis-DSM 34032 16S
CGAAGAGTTTGATCCTGGCTCAGGATGAACGCTAGCGACAGGCCTAACACATGCAAGTCGAGGGGCA
GCGGAGAGGTAGCAATACCTTTGCCGGCGACCGGCGCACGGGTGAGTAACACGTATGCAATCCACCT
GTAACAGGGGGATAACCCGGAGAAATCCGGACTAATACCCCATAATATGGGCTCTCCGCATGGAGGG
TCCATTAAAGAGAGCAATTTTGGTTACAGACGAGCATGCGCTCCATTAGCCAGTTGGCGGGGTAACGG
CCCACCAAAGCGACGATGGATAGGGGTTCTGAGAGGAAGGTCCCCCACATTGGAACTGAGACACGGT
CCAAACTCCTACGGGAGGCAGCAGTGAGGAATATTGGTCAATGGTCGGCAGACTGAACCAGCCAAGT
CGCGTGAGGGAAGACGGCCCTACGGGTTGTAAACCTCTTTTGTCGGAGAGTAAAGTACGCTACGTGTA
GCGTATTGCAAGTATCCGAAGAAAAAGCATCGGCTAACTCCGTGCCAGCAGCCGCGGTAATACGGAG
GATGCGAGCGTTATCCGGATTTATTGGGTTTAAAGGGTGCGTAGGCGGCACGCCAAGTCAGCGGTGAA
ATTTTCGGGCTCAACCCGGACTGTGCCGTTGAAACTGGCGAGCTAGAGTGCACAAGAGGCAGGCGGA
ATGCGTGGTGTAGCGGTGAAATGCATAGATATCACGCAGAACCCCGATTGCGAAGGCAGCCTGCTAG
GGTGCGACAGACGCTGAGGCACGAAAGCGTGGGTATCGAACAGGATTAGATACCCTGGTAGTCCACG
CAGTAAACGATGAATACTAACTGTTTGCGATACAATGTAAGCGGTACAGCGAAAGCGTTAAGTATTCC
ACCTGGGGAGTACGCCGGCAACGGTGAAACTCAAAGGAATTGACGGGGGCCCGCACAAGCGGAGGA
ACATGTGGTTTAATTCGATGATACGCGAGGAACCTTACCCGGGCTCAAACGCAGGGGGAATATATATG
AAAGTATATAGCTAGCAATAGTCACCTGCGAGGTGCTGCATGGTTGTCGTCAGCTCGTGCCGTGAGGT
GTCGGCTTAAGTGCCATAACGAGCGCAACCCCTATCGACAGTTACTAACGGGTCAAGCCGAGGACTCT
GTCGAGACTGCCGGCGCAAGCCGCGAGGAAGGTGGGGATGACGTCAAATCAGCACGGCCCTTACGTC
CGGGGCGACACACGTGTTACAATGGCAGGTACAGAAGGCAGCCAGTCAGCAATGACGCGCGAATCCC
GAAAACCTGTCTCAGTTCGGATTGGAGTCTGCAACCCGACTCCATGAAGCTGGATTCGCTAGTAATCG
CGCATCAGCCATGGCGCGGTGAATACGTTCCCGGGCCTTGTACACACCGCCCGTCAAGCCATGGAAGC
CGGGAGTACCTGAAGCATGCAACCGCAAGGAGCGTACGAAGGTAATACCGGTAACTGGGGCTAAGTC
GTAACAAGGTAGCCGTACCGGAAGGTGCGGCTGGAACACCTCCTTT (SEQ ID NO: 1)
Barnesiella intestinihominis-DSM 34012 16S
CGGCGACCGGCGCACGGGTGAGTAACACGTATGCAATCCACCTGTAACAGGGGGATAACCCGGAGAA
ATCCGGACTAATACCCCATAATATGGGCTCTCCGCATGGAGGGTCCATTAAAGAGAGCAATTTTGGTT
ACAGACGAGCATGCGCTCCATTAGCCAGTTGGCGGGGTAACGGCCCACCAAAGCGACGATGGATAGG
GGTTCTGAGAGGAAGGTCCCCCACATTGGAACTGAGACACGGTCCAAACTCCTACGGGAGGCAGCAG
TGAGGAATATTGGTCAATGGTCGGCAGACTGAACCAGCCAAGTCGCGTGAGGGAAGACGGCCCTACG
GGTTGTAAACCTCTTTTGTCGGAGAGTAAAGTACGCTACGCGTAGCGTATTGCAAGTATCCGAAGAAA
AAGCATCGGCTAACTCCGTGCCAGCAGCCGCGGTAATACGGAGGATGCGAGCGTTATCCGGATTTATT
GGGTTTAAAGGGTGCGTAGGCGGCACGCCAAGTCAGCGGTGAAATTTCCGGGCTCAACCCGGAATGT
GCCGTTGAAACTGGCGAGCTAGAGTGCACAAGAGGCAGGCGGAATGCGTGGTGTAGCGGTGAAATGC
ATAGATATCACGCAGAACCCCGATTGCGAAGGCAGCCTGCTAGGGTGCGACAGACGCTGAGGCACGA
AAGCGTGGGTATCGAACAGGATTAGATACCCTGGTAGTCCACGCAGTAAACGATGAATACTAACTGTT
TGCGATACAATGTAAGCGGTACAGCGAAAGCGTTAAGTATTCCACCTGGGGAGTACGCCGGCAACGG
TGAAACTCAAAGGAATTGACGGGGGCCCGCACAAGCGGAGGAACATGTGGTTTAATTCGATGATACG
CGAGGAACCTTACCCGGGCTCAAACGCAGGGGGAATATATATGAAAGTATATAGCTAGCAATAGTCA
CCTGCGAGGTGCTGCATGGTTGTCGTCAGCTCGTGCCGTGAGGTGTCGGCTTAAGTGCCATAACGAGC
GCAACCCCTATCGACAGTTACTAACGGGTCAAGCCGAGGACTCTGTCGAGACTGCCGGCGCAAGCCGC
GAGGAAGGTGGGGATGACGTCAAATCAGCACGGCCCTTACGTCCGGGGCGACACACGTGTTACAATG
GCAGGTACAGAAGGCAGCCAGTCAGCAATGACGCGCGAATCCCGAAAACCTGTCTCAGTTCGGATTG
GAGTCTGCAACCCGACTCCATGAAGCTGGATTCGCTAGTAATCGCGCATCAGCCATGGCGCGGTGAAT
ACGTTCCCGGGCCTTGTACACACCGCCCGTCAAGCCATGGAAGCCGGGAGTACCTGAAGCATGCAACC
GCAAGGAGCGTACGAAGGTAATACCGGTAACTGGGGCTAAGTCGTAACAAGGTAGCCGTACCGGAAG
GTGCGGCTGGAACACCTCCTTT (SEQ ID NO: 2)
Alistipes onderonkii -DSM 34033 16S
ATGGAGAGTTTGATCCTGGCTCAGGATGAACGCTAGCGGCAGGCCTAACACATGCAAGTCGAGGGGC
ATCACGAGGTAGCAATACTTTGGTGGCGACCGGCGCACGGGTGCGTAACGCGTATGTAACCTACCTAT
AACAGGGGCATAACACTGAGAAATTGGTACTAATTCCCCATAATATTCGGAGAGGCATCTCTCCGGGT
TGAAAACTCCGGTGGTTATAGATGGACATGCGTTGTATTAGCTAGTTGGTGAGGTAACGGCTCACCAA
GGCAACGATACATAGGGGGACTGAGAGGTTAACCCCCCACACTGGTACTGAGACACGGACCAGACTC
CTACGGGAGGCAGCAGTGAGGAATATTGGTCAATGGACGCAAGTCTGAACCAGCCATGCCGCGTGCA
GGAAGACGGCTCTATGAGTTGTAAACTGCTTTTGTACGAGGGTAAACTCACCTACGTGTAGGTGACTG
AAAGTATCGTACGAATAAGGATCGGCTAACTCCGTGCCAGCAGCCGCGGTAATACGGAGGATTCAAG
CGTTATCCGGATTTATTGGGTTTAAAGGGTGCGTAGGCGGTTTGATAAGTTAGAGGTGAAATCCCGGG
GCTTAACTCCGGAACTGCCTCTAATACTGTTAGACTAGAGAGTAGTTGCGGTAGGCGGAATGTATGGT
GTAGCGGTGAAATGCTTAGAGATCATACAGAACACCGATTGCGAAGGCAGCTTACCAAACTATATCTG
ACGTTGAGGCACGAAAGCGTGGGGAGCAAACAGGATTAGATACCCTGGTAGTCCACGCAGTAAACGA
TGATAACTCGTTGTCGGCGATACACAGTCGGTGACTAAGCGAAAGCGATAAGTTATCCACCTGGGGAG
TACGTTCGCAAGAATGAAACTCAAAGGAATTGACGGGGGCCCGCACAAGCGGAGGAACATGTGGTTT
AATTCGATGATACGCGAGAAACCTTACCCGGGCTTGAAAGTTACTGACGATTCTGGAAACAGGATTTC
CCTTTGGGGCAGGAAACTAGGTGCTGCATGGTTGTCGTCAGCTCGTGCCGTGAGGTGTCGGGTTAAGT
CCCATAACGAGCGCAACCCCTACCGTTAGTTGCCATCAGGTCAAGCTGGGCACTCTGGCGGGACTGCC
GGTGTAAGCCGAGAGGAAGGTGGGGATGACGTCAAATCAGCACGGCCCTTACGTCCGGGGCTACACA
CGTGTTACAATGGTAGGTACAGAGGGTCGCTACTCCGTGAGGAGATGCCAATCTCGAAAGCCTATCTC
AGTTCGGATTGGAGGCTGAAACCCGCCTCCATGAAGTTGGATTCGCTAGTAATCGCGCATCAGCCATG
GCGCGGTGAATACGTTCCCGGGCCTTGTACACACCGCCCGTCAAGCCATGGAAGCTGGGGGTGCCTGA
AGTTCGTGACCGCAAGGAGCGACCTAGGGCAAAACCGGTAACTGGGGCTAAGTCGTAACAAGGTAGC
CGTACCGGAAGGTGCGGCTGGAACACCTCCTTT (SEQ ID NO: 3)
Bacteroides finegoldii DSM 34013 16S
ATGAAGAGTTTGATCCTGGCTCAGGATGAACGCTAGCTACAGGCTTAACACATGCAAGTCGAGGGGCA
GCATTTTAGTTTGCTTGCAAACTAAAGATGGCGACCGGCGCACGGGTGAGTAACACGTATCCAACCTG
CCGATAACTCAGGGATAGCCTTTCGAAAGAAAGATTAATACCTGATGGCATAGGATTATCGCATGATA
ATCCTATTAAAGAATTTCGGTTATCGATGGGGATGCGTTCCATTAGGCAGTTGGTGAGGTAACGGCTC
ACCAAACCTTCGATGGATAGGGGTTCTGAGAGGAAGGTCCCCCACATTGGAACTGAGACACGGTCCA
AACTCCTACGGGAGGCAGCAGTGAGGAATATTGGTCAATGGACGGGAGTCTGAACCAGCCAAGTAGC
GTGAAGGATGACTGCCCTATGGGTTGTAAACTTCTTTTATACGGGAATAAAGTGATCCACGTGTGGGT
TTTTGTATGTACCGTATGAATAAGGATCGGCTAACTCCGTGCCAGCAGCCGCGGTAATACGGAGGATC
CGAGCGTTATCCGGATTTATTGGGTTTAAAGGGAGCGTAGGTGGATTGTTAAGTCAGTTGTGAAAGTT
TGCGGCTCAACCGTAAAATTGCAGTTGATACTGGCAGTCTTGAGTACAGTAGAGGTGGGCGGAATTCG
TGGTGTAGCGGTGAAATGCTTAGATATCACGAAGAACTCCGATTGCGAAGGCAGCTCACTGGACTGCA
ACTGACACTGATGCTCGAAAGTGTGGGTATCAAACAGGATTAGATACCCTGGTAGTCCACACAGTAAA
CGATGAATACTCGCTGTTTGCGATATACGGTAAGCGGCCAAGCGAAAGCGTTAAGTATTCCACCTGGG
GAGTACGCCGGCAACGGTGAAACTCAAAGGAATTGACGGGGGCCCGCACAAGCGGAGGAACATGTGG
TTTAATTCGATGATACGCGAGGAACCTTACCCGGGCTTAAATTACATTTGAATAATCTGGAAACAGGT
TAGCCGTAAGGCAAATGTGAAGGTGCTGCATGGTTGTCGTCAGCTCGTGCCGTGAGGTGTCGGCTTAA
GTGCCATAACGAGCGCAACCCTTATCTTCAGTTACTAACAGGTCATGCTGAGGACTCTGGAGAGACTG
CCGTCGTAAGATGTGAGGAAGGTGGGGATGACGTCAAATCAGCACGGCCCTTACGTCCGGGGCTACA
CACGTGTTACAATGGGGGGTACAGAAGGCAGCTACCTGGTGACAGGATGCTAATCCCAAAAACCTCTC
TCAGTTCGGATCGAAGTCTGCAACCCGACTTCGTGAAGCTGGATTCGCTAGTAATCGCGCATCAGCCA
TGGCGCGGTGAATACGTTCCCGGGCCTTGTACACACCGCCCGTCAAGCCATGAAAGCCGGGGGTACCT
GAAGTACGTAACCGCGAGGAGCGTCCTAGGGTAAAACTGGTAATTGGGGCTAAGTCGTAACAAGGTA
GCCGTACCGGAAGGTGCGGCTGGAACACCTCCTTT (SEQ ID NO: 4)
Bacteroides vulgatus DSM 34030 16S
ATGAAGAGTTTGATCCTGGCTCAGGATGAACGCTAGCTACAGGCTTAACACATGCAAGTCGAGGGGCA
GCATGGTCTTAGCTTGCTAAGGCCGATGGCGACCGGCGCACGGGTGAGTAACACGTATCCAACCTGCC
GTCTACTCTTGGACAGCCTTCTGAAAGGAAGATTAATACAAGATGGCATCATGAGTTCACATGTTCAC
ATGATTAAAGGTATTCCGGTAGACGATGGGGATGCGTTCCATTAGATAGTAGGCGGGGTAACGGCCCA
CCTAGTCTTCGATGGATAGGGGTTCTGAGAGGAAGGTCCCCCACATTGGAACTGAGACACGGTCCAAA
CTCCTACGGGAGGCAGCAGTGAGGAATATTGGTCAATGGGCGCAGGCCTGAACCAGCCAAGTAGCGT
GAAGGATGACTGCCCTATGGGTTGTAAACTTCTTTTATAAAGGAATAAAGTCGGGTATGTATACCCGT
TTGCATGTACTTTATGAATAAGGATCGGCTAACTCCGTGCCAGCAGCCGCGGTAATACGGAGGATCCG
AGCGTTATCCGGATTTATTGGGTTTAAAGGGAGCGTAGATGGATGTTTAAGTCAGTTGTGAAAGTTTG
CGGCTCAACCGTAAAATTGCAGTTGATACTGGATATCTTGAGTGCAGTTGAGGCAGGCGGAATTCGTG
GTGTAGCGGTGAAATGCTTAGATATCACGAAGAACTCCGATTGCGAAGGCAGCCTGCTAAGCTGCAAC
TGACATTGAGGCTCGAAAGTGTGGGTATCAAACAGGATTAGATACCCTGGTAGTCCACACGGTAAACG
ATGAATACTCGCTGTTTGCGATATACAGCAAGCGGCCAAGCGAAAGCGTTAAGTATTCCACCTGGGGA
GTACGCCGGCAACGGTGAAACTCAAAGGAATTGACGGGGGCCCGCACAAGCGGAGGAACATGTGGTT
TAATTCGATGATACGCGAGGAACCTTACCCGGGCTTAAATTGCAGATGAATTACGGTGAAAGCCGTAA
GCCGCAAGGCATCTGTGAAGGTGCTGCATGGTTGTCGTCAGCTCGTGCCGTGAGGTGTCGGCTTAAGT
GCCATAACGAGCGCAACCCTTGTTGTCAGTTACTAACAGGTTCCGCTGAGGACTCTGACAAGACTGCC
ATCGTAAGATGTGAGGAAGGTGGGGATGACGTCAAATCAGCACGGCCCTTACGTCCGGGGCTACACA
CGTGTTACAATGGGGGGTACAGAGGGCCGCTACCACGCGAGTGGATGCCAATCCCAAAAACCTCTCTC
AGTTCGGACTGGAGTCTGCAACCCGACTCCACGAAGCTGGATTCGCTAGTAATCGCGCATCAGCCACG
GCGCGGTGAATACGTTCCCGGGCCTTGTACACACCGCCCGTCAAGCCATGGGAGCCGGGGGTACCTGA
AGTGCGTAACCGCGAGGAGCGCCCTAGGGTAAAACTGGTGACTGGGGCTAAGTCGTAACAAGGTAGC
CGTACCGGAAGGTGCGGCTGGAACACCTCCTTT (SEQ ID NO: 5)
Oscillibacter sp.-DSM 34011 16S
TATAGAGAGTTTGATCCTGGCTCAGGACGAACGCTGGCGGCGTGCTTAACACATGCAAGTCGAACGGA
GCACCCTTGATTGAGGTTTCGGCCAAATGATAGGAATGCTTAGTGGCGGACTGGTGAGTAACGCGTGA
GGAACCTGCCTTTCAGAGGGGGACAACAGTTGGAAACGACTGCTAATACCGCATGACGCATTTTGATC
GCATGGTCGAAATGCCAAAGATTTATCGCTGAAAGATGGCCTCGCGTCTGATTAGATAGTTGGCGGGG
TAACGGCCCACCAAGTCGACGATCAGTAGCCGGACTGAGAGGTTGACCGGCCACATTGGGACTGAGA
TACGGCCCAGACTCCTACGGGAGGCAGCAGTGGGGAATATTGGGCAATGGGCGCAAGCCTGACCCAG
CAACGCCGCGTGAAGGAAGAAGGCTTTCGGGTTGTAAACTTCTTTTAAGTGGGAAGAGTAGAAGACG
GTACCACTTGAATAAGCCACGGCTAACTACGTGCCAGCAGCCGCGGTAATACGTAGGTGGCAAGCGTT
GTCCGGATTTACTGGGTGTAAAGGGCGTGCAGCCGGGCATGCAAGTCAGATGTGAAATCTCAGGGCTT
AACCCTGAAACTGCATTTGAAACTGTATGTCTTGAGTGCCGGAGAGGTAATCGGAATTCCTTGTGTAG
CGGTGAAATGCGTAGATATAAGGAAGAACACCAGTGGCGAAGGCGGATTACTGGACGGTAACTGACG
GTGAGGCGCGAAAGCGTGGGGAGCGAACAGGATTAGATACCCTGGTAGTCCACGCTGTAAACGATGG
ATACTAGGTGTGCGGGGACTGACCCCCTGCGTGCCGCAGTTAACACAATAAGTATCCCACCTGGGGAG
TACGATCGCAAGGTTGAAACTCAAAGGAATTGACGGGGGCCCGCACAAGCGGTGGATTATGTGGTTTA
ATTCGAAGCAACGCGAAGAACCTTACCAGGTCTTGACATCCTGCTAACGAAGTAGAGATACATTAGGT
GCCCTTCGGGGAAAGCAGAGACAGGTGGTGCATGGTTGTCGTCAGCTCGTGTCGTGAGATGTTGGGTT
AAGTCCCGCAACGAGCGCAACCCCTATTGTTAGTTGCTACGCAAGAGCACTCTAGCGAGACTGCCGTT
GACAAAACGGAGGAAGGTGGGGACGACGTCAAATCATCATGCCCCTTATGACCTGGGCTACACACGT
AATACAATGGCGGTCAACAAAGGGATGCAAAGCCGCGAGGCAGAGCGAACCCCAAAAAGCCGTCCCA
GTTCGGATCGCAGGCTGCAACCCGCCTGCGTGAAGTCGGAATCGCTAGTAATCGTGGGTCAGCATACC
ACGGTGAATACGTTCCCGGGCCTTGTACACACCGCCCGTCACACCATGAGAGTCGGGAACACCCGAAG
TCCGTAGCCTAACCGCAAGGAGGGCGCGGCCGAAGGTGGGTTCGATAATTGGGGTGAAGTCGTAACA
AGGTAGCCGTTCGAGAACGAGCGGCTGGATCACCTCCTTT (SEQ ID NO: 6)

We claim:

1. A composition comprising at least one or more of (a) a biologically pure strain of Barnesiella intestinihominis; (b) a biologically pure strain of Alistipes onderdonkii; (c) a biologically pure strain of Bacteroides finegoldii; (d) a biologically pure strain of Bacteroides vulgatus; and/or (e) a bacterial strain having a 16S ribosomal RNA sequence displaying at least 97.0% sequence similarity to a 16S ribosomal RNA sequence of an Oscillibacter sp. deposited at the German Collection of Microorganisms and Cell Cultures (DSM) under number DSM 34011.

2. The composition of claim 1, comprising (a)(i) a bacterial strain having a 16S ribosomal RNA sequence displaying at least 97.0% sequence similarity to a 16S ribosomal RNA sequence of Barnesiella intestinihominis deposited at DSM under number DSM 34032; and/or (ii) a bacterial strain having a 16S ribosomal RNA sequence displaying at least 97.0% sequence similarity to a 16S ribosomal RNA sequence of Barnesiella intestinihominis deposited at DSM under number DSM 34012; (b) a bacterial strain having a 16S ribosomal RNA sequence displaying at least 97.0% sequence similarity to a 16S ribosomal RNA sequence of Alistipes onderdonkii deposited at DSM under number DSM 34033; (c) a bacterial strain having a 16S ribosomal RNA sequence displaying at least 97.0% sequence similarity to a 16S ribosomal RNA sequence of Bacteroides finegoldii deposited at DSM under number DSM 34013; and/or (d) a bacterial strain having a 16S ribosomal RNA sequence displaying at least 97.0% sequence similarity to a 16S ribosomal RNA sequence of Bacteroides vulgatus deposited at DSM under number DSM 34030.

3. The composition of claim 1 or claim 2, wherein (a)(i) the 16S ribosomal RNA sequence of Barnesiella intestinihominis deposited at DSM under number DSM 34032 comprises SEQ ID NO:1; (ii) the 16S ribosomal RNA sequence displaying at least 97.0% sequence similarity to a 16S ribosomal RNA sequence of Barnesiella intestinihominis deposited at DSM under number DSM 34012 comprises SEQ ID NO:2; (b) the 16S ribosomal RNA sequence of Alistipes onderdonkii deposited at DSM under number DSM 34033 comprises SEQ ID NO:3; (c) the 16S ribosomal RNA sequence of Bacteroides finegoldii deposited at DSM under number DSM 34013comprises SEQ ID NO:4; (d) the 16S ribosomal RNA sequence displaying at least 97.0% sequence similarity to a 16S ribosomal RNA sequence of Bacteroides vulgatus deposited at DSM under number DSM 34030 comprises SEQ ID NO:5; and/or (c) the 16S ribosomal RNA sequence of an Oscillibacter sp. deposited at the German Collection of Microorganisms and Cell Cultures (DSM) under number DSM 34011 comprises SEQ ID NO:6.

4. The composition of any one of claims 1-3, comprising (a)(i) the Barnesiella intestinihominis strain deposited at DSM under number DSM 34032 or a live strain having all of the identifying characteristics of the Barnesiella intestinihominis strain deposited at DSM under number DSM 34032; and/or (ii) the Barnesiella intestinihominis strain deposited at DSM under number DSM 34012 or a live strain having all of the identifying characteristics of the B. intestinihominis strain deposited at DSM under number DSM 34012; (b) the Alistipes onderdonkii strain deposited at DSM under number DSM 34033 or a live strain having all of the identifying characteristics of the Alistipes onderdonkii strain deposited at DSM under number DSM 34033; (c) the Bacteroides finegoldii strain deposited at DSM under number DSM 34013 or a live strain having all of the identifying characteristics of the Bacteroides finegoldii strain deposited at DSM under number DSM 34013; (d) the Bacteroides vulgatus strain deposited at DSM under number DSM 34030 or a live strain having all of the identifying characteristics of the Bacteroides vulgatus strain deposited at DSM under number DSM 34030; and/or (c) the Oscillibacter sp. deposited at DSM under number DSM 34011 or a live strain having all of the identifying characteristics of the Oscillibacter sp. deposited at DSM under number DSM 34011 either (A) alone; and/or (B) in combination with a culture supernatant derived from one or more of these strains.

5. A composition comprising isolated bacterial extracellular vesicles (EVs) derived from at least one or more of (a) a biologically pure strain of Barnesiella intestinihominis; (b) a biologically pure strain of Alistipes onderdonkii; (c) a biologically pure strain of Bacteroides finegoldii; (d) a biologically pure strain of Bacteroides vulgatus; and/or (e) a bacterial strain having a 16S ribosomal RNA sequence displaying at least 97.0% sequence similarity to a 16S ribosomal RNA sequence of an Oscillibacter sp. deposited at the German Collection of Microorganisms and Cell Cultures (DSM) under number DSM 34011.

6. The composition of claim 5, further comprising one or more bacteria from (a), (b), (c), (d), and/or (c).

7. The composition of claim 5 or claim 6, comprising (a)(i) EVs derived from a bacterial strain having a 16S ribosomal RNA sequence displaying at least 97.0% sequence similarity to a 16S ribosomal RNA sequence of Barnesiella intestinihominis deposited at DSM under number DSM 34032; and/or (ii) EVs derived from a bacterial strain having a 16S ribosomal RNA sequence displaying at least 97.0% sequence similarity to a 16S ribosomal RNA sequence of Barnesiella intestinihominis deposited at DSM under number DSM 34012; (b) EVs derived from a bacterial strain having a 16S ribosomal RNA sequence displaying at least 97.0% sequence similarity to a 16S ribosomal RNA sequence of Alistipes onderdonkii deposited at DSM under number DSM 34033; (c) EVs derived from a bacterial strain having a 16S ribosomal RNA sequence displaying at least 97.0% sequence similarity to a 16S ribosomal RNA sequence of Bacteroides finegoldii deposited at DSM under number DSM 34013; and/or (d) EVs derived from a bacterial strain having a 16S ribosomal RNA sequence displaying at least 97.0% sequence similarity to a 16S ribosomal RNA sequence of Bacteroides vulgatus deposited at DSM under number DSM 34030.

8. The composition of any one of claims 5-7, wherein (a)(i) the 16S ribosomal RNA sequence of Barnesiella intestinihominis deposited at DSM under number DSM 34032 comprises SEQ ID NO:1; (ii) the 16S ribosomal RNA sequence displaying at least 97.0% sequence similarity to a 16S ribosomal RNA sequence of Barnesiella intestinihominis deposited at DSM under number DSM 34012 comprises SEQ ID NO:2; (b) the 16S ribosomal RNA sequence of Alistipes onderdonkii deposited at DSM under number DSM 34033 comprises SEQ ID NO:3; (c) the 16S ribosomal RNA sequence of Bacteroides finegoldii deposited at DSM under number DSM 34013 comprises SEQ ID NO:4; (d) the 16S ribosomal RNA sequence displaying at least 97.0% sequence similarity to a 16S ribosomal RNA sequence of Bacteroides vulgatus deposited at DSM under number DSM 34030 comprises SEQ ID NO:5; and/or (e) the 16S ribosomal RNA sequence of an Oscillibacter sp. deposited at the German Collection of Microorganisms and Cell Cultures (DSM) under number DSM 34011 comprises SEQ ID NO:6.

9. The composition of any one of claims 5-8, comprising (a)(i) EVs derived from the Barnesiella intestinihominis strain deposited at DSM under number DSM 34032 or a live strain having all of the identifying characteristics of the Barnesiella intestinihominis strain deposited at DSM under number DSM 34032; and/or (ii) EVs derived from the Barnesiella intestinihominis strain deposited at DSM under number DSM 34012 or a live strain having all of the identifying characteristics of the B. intestinihominis strain deposited at DSM under number DSM 34012; (b) EVs derived from the Alistipes onderdonkii strain deposited at DSM under number DSM 34033 or a live strain having all of the identifying characteristics of the Alistipes onderdonkii strain deposited at DSM under number DSM 34033; (c) EVs derived from the Bacteroides finegoldii strain deposited at DSM under number DSM 34013 or a live strain having all of the identifying characteristics of the Bacteroides finegoldii strain deposited at DSM under number DSM 34013; (d) EVs derived from the Bacteroides vulgatus strain deposited at DSM under number DSM 34030 or a live strain having all of the identifying characteristics of the Bacteroides vulgatus strain deposited at DSM under number DSM 34030; and/or (e) EVs derived from the Oscillibacter sp. deposited at DSM under number DSM 34011 or a live strain having all of the identifying characteristics of the Oscillibacter sp. deposited at DSM under number DSM 34011.

10. The composition of any one of claims 1-9, wherein the composition is lyophilized or freeze dried.

11. The composition of any one of claims 1-10, wherein the composition is encapsulated or coated.

12. The composition of claim 11, wherein the coating is an enteric coating.

13. The composition of any one of claims 1-12, wherein the composition is a food product, food ingredient, dietary supplement, or medicament.

14. The composition of any one of claims 1-13, wherein at least about 1×10⁴ CFU/g composition to at least about 1×10¹² CFU/g composition of bacteria is present in the composition.

15. The composition of any one of claims 1-14, wherein the composition is a probiotic.

16. The composition of any one of claims 1-15, wherein the composition has been pasteurized or heat treated.

17. The composition of any one of claims 1-16, wherein the composition is a pharmaceutical composition and further comprises at least one pharmaceutically acceptable carrier and/or excipient.

18. The composition of any one of claims 1-17, further comprising one or more additional probiotic microorganisms and/or a prebiotic.

19. The composition of claim 18, wherein at least one of the one or more additional probiotic microorganisms is listed on Table 1.

20. A tablet, prolonged-release capsule, prolonged-release granule, powder, sachet, or gummy comprising the composition of any one of claims 1-19.

21. A kit comprising (a)(i) the composition of any one of claims 1-19; or (ii) the tablet, prolonged-release capsule, prolonged-release granule, powder, sachet, or gummy of claim 20 and b) written instructions for administration to a subject.

22. A method for treating and/or preventing one or more obesity related disorders in a subject in need thereof, comprising administering a therapeutically effective amount of the composition of any one of claims 1-19 or the tablet, prolonged-release capsule, prolonged-release granule, powder, sachet, or gummy of claim 20 to the subject.

23. The method of claim 21, wherein the obesity related disorder is one or more disorders selected from the group consisting of obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease.

24. The method of claim 23, wherein cardiovascular disease is one or more of coronary heart disease, cardiomyopathy, ischemic heart disease, heart failure, peripheral arterial disease, hypertension, inflammatory heart disease, valvular heart disease and/or aneurysm.

25. The method of any one of claims 22-24, wherein the method results in increased production of one or more of agmatine, carnosine, Ile-Pro-Pro and Val-Pro-Pro bioactive tripeptides, octanoic acid, short chain fatty acids, and/or branched-chain keto acids in the subject.

26. A method for treating and/or preventing viral infection in a subject in need thereof, comprising administering a therapeutically effective amount of the composition of any one of claims 1-19 or the tablet, prolonged-release capsule, prolonged-release granule, powder, sachet, or gummy of claim 20 to the subject.

27. The method of claim 26, wherein the method results in increased production of penciclovir in the subject.

28. A composition for use in the prevention and/or treatment of one or more obesity-related disorders in a subject in need thereof, comprising the composition of any one of claims 1-19 or the tablet, prolonged-release capsule, prolonged-release granule, powder, sachet, or gummy of claim 20 to the subject.

29. The composition for use of claim 28, wherein the obesity related disorder is one or more disorders selected from the group consisting of obesity, metabolic syndrome, diabetes mellitus, insulin deficiency-related disorders, insulin-resistance related disorders, glucose intolerance, abnormal lipid metabolism, non-alcoholic fatty liver disease, hepatic steatosis, leptin resistance, reduced resistin levels, and/or cardiovascular disease.

30. The composition for use of claim 28 or claim 29, wherein cardiovascular disease is one or more of coronary heart disease, cardiomyopathy, ischemic heart disease, heart failure, peripheral arterial disease, hypertension, inflammatory heart disease, valvular heart disease and/or aneurysm.

31. The composition for use of any one of claims 28-30, wherein the composition results in increased production of one or more of agmatine, carnosine, Ile-Pro-Pro and Val-Pro-Pro bioactive tripeptides, octanoic acid, short chain fatty acids, and/or branched-chain keto acids in the subject.

32. A composition for use in the prevention and/or treatment of viral infection in a subject in need thereof, comprising the composition of any one of claims 1-19 or the tablet, prolonged-release capsule, prolonged-release granule, powder, sachet, or gummy of claim 20 to the subject.

33. The composition for use of claim 32, wherein the composition results in increased production of penciclovir in the subject.
